(19) United States
(12) Patent Application Publication
(10) Pub. No.: US 2015/0320295 A1
(43) Pub. Date: Nov. 12, 2015

(54) ACTIVATED POLYMER ARTICULATED INSTRUMENTS AND METHODS OF INSERTION

(71) Applicant: INTUITIVE SURGICAL OPERATIONS, INC., Sunnyvale, CA (US)

(72) Inventors: Amir BELSON, Sunnyvale, CA (US); Robert M. OHLINE, Redwood City, CA (US)

(73) Assignee: INTUITIVE SURGICAL OPERATIONS, INC., Sunnyvale, CA (US)

(21) Appl. No.: 14/449,925

(22) Filed: Aug. 1, 2014

Related U.S. Application Data
(63) Continuation of application No. 11/481,483, filed on Jul. 5, 2006, now abandoned.
Application No. 11/481,483 is a continuation of application No. 10/923,602, filed on Aug. 20, 2004, now abandoned.
Application No. 10/923,602 is a continuation-in-part of application No. 10/622,801, filed on Jul. 18, 2003, now abandoned.
Application No. 10/622,801 is a continuation of application No. 09/969,927, filed on Oct. 2, 2001, now Pat. No. 6,610,007.
Application No. 09/969,927 is a continuation-in-part of application No. 09/790,204, filed on Feb. 20, 2001, now Pat. No. 6,468,203.
Application No. 09/790,204 is a continuation-in-part of application No. 10/228,583, filed on Aug. 26, 2002, now Pat. No. 6,869,396.
Application No. 10/228,583 is a continuation of application No. 09/790,204, filed on Feb. 20, 2001, now Pat. No. 6,468,203.
(60) Provisional application No. 60/194,140, filed on Apr. 3, 2000.
(60) Provisional application No. 60/496,943, filed on Aug. 20, 2003.
(60) Provisional application No. 60/194,140, filed on Apr. 3, 2000.

(51) Int. Cl. A61B 1/008; H01L 41/193; A61B 1/31; H01L 41/09; A61B 1/00; A61B 1/005

(52) U.S. Cl. CPC A61B 1/008; A61B 1/00006; A61B 1/0055; A61B 1/31; H01L 41/0926; H01L 41/193

(57) ABSTRACT

An electro-polymeric articulated endoscope and method of insertion are described herein. A steerable endoscope having a segmented, elongated body with a manually or selectively steerable distal portion and an automatically controlled proximal portion can be articulated by electro-polymeric materials. These materials are configured to mechanically contract or expand in the presence of a stimulus, such as an electrical field. Adjacent segments of the endoscope can be articulated using the electro-polymeric material by inducing relative differences in size or length of the material when placed near or around the outer periphery along a portion of the endoscope.

### CROSS REFERENCE TO RELATED APPLICATIONS

The present application is a continuation of U.S. patent application Ser. No. 10/923,602, filed Aug. 20, 2004, which is a continuation-in-part of U.S. patent application Ser. No. 10/228,583, filed Aug. 26, 2002, which is a continuation of U.S. patent application Ser. No. 09/790,204 entitled “Steerable Endoscope and Improved Method of Insertion” filed Feb. 20, 2001 (now U.S. Pat. No. 6,468,203), which claims priority to U.S. Provisional Patent Application No. 60/194,140 filed Apr. 3, 2000; and a continuation in part of U.S. patent application Ser. No. 10/622,801 filed Jul. 13, 2003, which is a continuation of U.S. patent application Ser. No. 09/969,927 entitled “Steerable Segmented Endoscope and Method of Insertion” filed Oct. 2, 2001 (now U.S. Pat. No. 6,610,007) which is a continuation in part of application Ser. No. 09/790,204 filed Feb. 20, 2001 (now U.S. Pat. No. 6,468,203) which claims priority of U.S. Provisional Patent Application No. 60/194,140 filed Apr. 3, 2000; and claims priority to U.S. Provisional Patent Application No. 60/496,943 filed Aug. 20, 2003, each of which is incorporated herein by reference in their entirety.

### FIELD OF THE INVENTION

The present invention relates generally to articulating instruments and the use of such instruments. More particularly, it relates to articulating instruments, methods and devices that advantageously utilize plastic electromechanical actuators to facilitate insertion and control of articulating instruments along selected pathways in industrial and medical settings.

### BACKGROUND OF THE INVENTION

There are numerous examples of articulating or bendable or steerable instruments used in a wide variety of industrial and medical applications. In general, the articulating instrument is directed to advance along a selected or desired pathway to accomplish a task such as inspection, repair, etc. The more convoluted the pathway, the higher degree of articulation, control, and flexibility needed to maneuver the instrument into the desired position. As the degree of movement and control for an articulating instrument increases, the number, variety and size of actuator components needed to operate the instrument may increase as well.

Articulating instruments find use in a wide variety of commercial settings including, for example, industrial robotic applications and medical applications. One example of an articulating medical instrument is an endoscope. An endoscope is a medical instrument for visualizing the interior of a patient's body. Endoscopes are used for a variety of different diagnostic and interventional procedures, including colonoscopy, bronchoscopy, thoracoscopy, laparoscopy and video endoscopy. The desire to access remote portions of the body more efficiently or access one area of the body while avoiding other areas along the way results increases the complexity of articulating endoscopes and articulating surgical instruments generally.

Insertion of the colonoscope is complicated by the fact that the colon represents a tortuous and convoluted path. Considerable manipulation of the colonoscope is often necessary to advance the colonoscope through the colon, making the procedure more difficult and time consuming and adding to the potential for complications, such as intestinal perforation. Steerable colonoscopes have been devised to facilitate selection of the correct path though the curves of the colon. However, as the colonoscope is inserted farther into the colon, it becomes more difficult to advance the colonoscope along the selected path. Only the distal tip of a standard colonoscope is steerable, typically 10 cm in length, and the remainder of the colonoscope body is passive. The performance of the device is therefore limited. Push forces imparted to the colonoscope by a physician or other user do not result in forward movement of the colonoscope tip if the shape of the colonoscope body has assumed a complex curve within the colon. After a complex curve has developed, with more than one bend in any plane, push forces on the proximal end of the colonoscope result in the enlargement of the device's most proximal curve. This results in “looping” of the colonoscope, in which the most proximal curve defined by the colonoscope enlarges and the distal tip of the instrument fails to advance further into the colon.

At each turn, the wall of the colon must maintain the curve in the colonoscope. The colonoscope rubs against the mucosal surface of the colon along the outside of each turn. Friction and slack in the colonoscope build up at each turn, making it more and more difficult to advance and withdraw, and can result in looping of the colonoscope. In addition, the force against the wall of the colon increases with the buildup of friction. In cases of extreme tortuosity, it may become impossible to advance the colonoscope all of the way through the colon.

A variety of electromechanical actuators based on the principal that certain types of polymers can change shape under certain conditions of stimulation have been under investigation for decades. This research was organized by Yoseph Bar-Cohen in a book entitled “Electroactive Polymer (EAP) Actuators as Artificial Muscles: Reality, Potential and Challenges” (SPIE Press, January 2001). As used herein, activated polymer refers generally to the families of polymers described by Bar-Cohen. More precision is needed to accurately describe what type of polymer is actually under examination. It is useful to classify these polymers by their mode of activation. As suggested by Bar-Cohen, these would include: non-electrically actuated polymers, ionically actuated polymers and electrically actuated polymers. There are numerous subcategories within each type of activation mechanism. According to Bar-Cohen, ionically actuated polymers include electroactive polymer gels, ionomeric polymer-metal composites, conductive polymers, and carbon nanotubes.

Couvillon et al have suggested some uses for conductive polymer actuators (i.e., US Patent Application Ser Nr. US 2003/0069474). Couvillon et al describes conducting polymers as a class of polymers having a conjugated backbone and which are electrically conductive. Couvillon lists polyaniline, polypyrrole, and polyacetylene as examples of conductive polymers. Bar-Cohen and others also categorize each of these materials as conductive polymers.

Conductive polymers, such as those described by Couvillon et al., suffer from a number of drawbacks that limit their utility for use as actuators for articulating instruments. The activation mechanism of a conductive polymer actuator is based on an ion exchange process between the conductive polymer film and the electrolytic medium. According to Bar-Cohen, this is the factor that controls and limits the response time of a conductive polymer actuator. Response time can be improved through the use of gel or liquid electrolyte, however this alternative requires that the actuator be encapsulated. On the other hand, solid electrolytes do not require encapsulation but have low ionic conductivity and may or may not have low enough mechanical stiffness to operate effectively with articulating instruments.

Another challenge facing those who suggest using conductive polymers are the materials themselves. Conductive polymers are r-conjugated systems where single and double bonds alternate along the polymer chains. These polymers are not inherently conductive but are instead transformed into conductive polymers using a process called “doping” to chemically or electrochemically modify the structure and conductivity of the polymer. Numerous challenges exist in the doping process and the maintenance of the conductive state after numerous reduction/oxidation reaction cycles. Moreover, conjugated polymers are not chemically stable and their charging capacity gradually declines when they are cycled. Yet another challenge facing conductive polymers is delamination at the electrode/conductive polymer interface. In 1999, Smela et al. reported delamination as the failure mode of a conductive polymer actuator using polypyrrole with gold electrodes (Bar-Cohen, pg. 206).

Given the above listed and other challenges and shortcomings of conductive polymers, there remains a need for articulating instruments that more fully realize the advantages of activated polymers and activated polymer based actuators.

### BRIEF SUMMARY OF THE INVENTION

In some embodiments of the present invention there are provided articulating instruments for use in a wide variety of medical and industrial applications. In one aspect, articulating instruments have a plurality of controllable segments that provide for the articulation of the instrument. Some of the segments are steerable or controllable by a user (with or without computer controlled assistance) into or along a selected or desired pathway while others are electronically or computer controlled to follow the shape of the previously steered segments in a so called “follow the leader” manner. The “follow the leader” technique is described in the commonly owned and co-pending U.S. patent application (pending Belson '203 application). In aspects of the invention, controlling a segment refers to the activation of selected electromechanical actuators to position a segment or plurality of segments into a desired shape. In other aspects of the invention, controlling refers not only to the activation of selected electromechanical actuators to position a segment or plurality of segments into a desired shape but also the use of an electronic, computer based or other known motion controller to propagate the selected shape to other segments as those segments advance distally or proximally.

In some aspects, the articulating instrument is a steerable endoscope for the examination of a patient's colon, other internal bodily cavities, or other internal body spaces with minimal impingement upon the walls of those organs. In one aspect, the steerable endoscope described herein has a segmented, elongated body with a manually or selectively steerable distal portion (at least one segment) and an automatically controlled proximal portion. In a further aspect, the selectively steerable distal portion can be flexed in any direction relative to the rest of the device, e.g., by controlling the arc lengths on opposing sides of the walls or circumferential periphery of said distal portion or otherwise providing actuation forces that alter the relative geometry or relationship between segments.

In one aspect, the selectively steerable distal portion can be selectively steered (or bent) up to, e.g., a full 180 degrees, in any direction relative to the rest of the device. A fiberoptic imaging bundle and one or more illumination fibers may extend through the body from the proximal portion to the distal portion. The illumination fibers are preferably in communication at its proximal end with a light source, e.g., conventional light sources such as incandescent lights, which may be positioned at some location external to the device and/or the patient, or other sources such as LEDs. Alternatively, the endoscope may be configured as a video endoscope with a miniature video camera, such as a CCD or CMOS camera, positioned at the distal portion of the endoscope body. The video camera may be used in combination with the illumination fibers. Optionally, the body of the endoscope may also include one or two access lumens that may be used, for example, for: insufflation or irrigation, air and water channels, and vacuum channels, etc. Generally, the body of the endoscope is highly flexible so that it is able to bend around small diameter curves without buckling or kinking while maintaining the various channels intact. The endoscope can be made in a variety of sizes and configurations for other medical and industrial applications.

In another aspect, the steerable distal portion of the endoscope may be first advanced through an opening into the patient's body, e.g., into the rectum via the anus, through a stoma in the case of a colostomy procedure, etc. The endoscope may be simply advanced, either manually or automatically by a motor or some other method of actuation, until the first curvature of the patient's gastrointestinal tract is reached. At this point, the user (e.g., a physician or surgeon) can actively control the steerable distal portion to attain an optimal curvature or shape for advancement of the endoscope. The optimal curvature or shape is generally the path that presents the least amount of contact or interference from the walls of the colon. In one variation, once the desired curvature has been determined, the endoscope may be advanced further into the colon such that the automatically controlled segments of the controllable portion follow the distal portion while transmitting the optimal curvature or shape proximally down the remaining segments of the controllable portion. Thus, as the instrument is advanced, it follows the path that the distal portion has defined. A more detailed description of one variation for insertion of the endoscopic device may be seen in co-owned U.S. Pat. No. 6,468,203, which is incorporated herein by reference in its entirety. The operation of the controllable segments will be described in further detail below.

In one aspect of the invention, actuation of the articulating instrument is accomplished by an electromechanical actuator that includes a plastic actuator such as those based on the activation of a polymer. In one aspect, the electromechanical actuator including a plastic actuator where the polymer is a non-electrically activated polymer. In another aspect, the electromechanical actuator including a plastic actuator where the polymer is an ionically activated polymer. In another aspect, the electromechanical actuator including a plastic actuator where the polymer is activated using Coulomb forces. In another aspect, the electromechanical actuator including a plastic actuator where the polymer is activated using electrical forces. In another aspect, the electromechanical actuator including a plastic actuator where the polymer is actuated using forces, alone or in combination, such as electrostrictive, electrostatic, piezoelectric and/or ferroelectric.

In one aspect, the invention provides an articulating instrument having controllable segments actuated or manipulated through the controlled use of an ionically activated polymer electromechanical actuator incapable of sustaining an activated condition using a dc bias. In one aspect, the invention provides an articulating instrument that is actuated or manipulated through the controlled use of an ionically activated polymer actuator activated without the use of an electrolyte. In a further aspect, the ionically activated polymer actuator comprises an electroactive polymer gel. In a further aspect, the ionically activated polymer gel actuator comprises a physical gel, a chemical gel, a chemically actuated gel, or an electrically actuated gel. In a further aspect, the ionically activated polymer actuator comprises an ionomeric polymer-metal composite. In a further aspect, the ionically activated polymer actuator comprises a carbon nanotube. In a further aspect, the ionically activated polymer actuator activates resulting in movement of the articulating instrument without the ionically activated polymer undergoing an oxidation/reduction process.

In another aspect, the invention provides an articulating instrument having controllable segments actuated or manipulated through the controlled use of an electromechanical actuator consisting essentially of a polymer and a pair of compliant electrodes coupled to the polymer thereby forming an active area on the polymer that is used to control or manipulate the articulating instrument.

In another aspect, the invention provides an articulating instrument having controllable segments actuated or manipulated through the controlled use of an conductive polymer actuator having a conductive polymer in contact with an electrolytic media and electrical energy provided into the conductive polymer and the electrolytic media via at least one pair of compliant electrodes.

In another aspect, the invention provides an articulating instrument having controllable segments actuated or manipulated through the controlled use of an electromechanical actuator comprising a dielectric polymer, a pair of electrodes forming an active area with the polymer, the deflections of the polymer in the active area being used to control or manipulate the articulating instrument. In a further aspect, the invention provides a plurality of electrode pairs forming a plurality of active areas that are synergistically controlled to manipulate the articulating instrument. In a further aspect, the electrodes are compliant electrodes.

In a further aspect, the invention provides an articulating instrument that is actuated or manipulated through use of an electromechanical actuator from the category of an electronic electroactive polymer based actuator. In one aspect, an electronic electroactive polymer based actuator is used to articulate the controllable segments of an endoscope, including the distal steerable portion. In another aspect, embodiments of the electronic electroactive polymer based actuator include, but are not limited to, non-doped polymers, dielectric elastomers, electrostatically stricted polymers, electrostrictor polymer (i.e., polyvinylidene fluoride-triflouroethylene copolymer or P(VDF-TrFE)), polyurethane (such as manufactured by Deerfield: PT6100S), silicone (such as manufactured by Dow Corning: Sylgard 186), fluorosilicone (such as manufactured by Dow Corning: 730), fluoroelastomer (such as manufactured by LaurenL143HC), polybutadiene (such as manufactured by Aldrich: PBD), isoprene natural rubber latex, acrylic, acrylic elastomer, pre-strained dielectric elastomer, acrylic electroactive polymer artificial muscle, silicone (CF19-2186) electroactive polymer artificial muscle.

In another aspect, the plastic actuator is formed using laminate polymer sheet structures including combinations strained polymers, unstained polymers, compliant electrodes, active areas creating one planar direction of polymer deformation, active areas creating two planar directions of polymer deformation, compliant electrode patterning that produces multiple degrees of freedom and combinations of the above.

In other aspects of the invention, the plastic electromechanical actuator relies on actuation from other materials, for example, infused mixtures of polymer gels with or without electrorheological fluid, electrorheological fluid, polydimethyl siloxane, polyacrylonitrile, carbon nanotubes and carbon single-wall nanotubes (SWNT).

In another aspect, there is provided a method of advancing along a path an instrument having a plurality of selectively controllable segments, a plurality of automatically controllable segments, an electronic motion controller, and a plastic actuator connected to each segment to alter the geometry of the segment under the control of the electronic motion controller, including selectively altering the geometry of a selectively controllable segment to assume a curve along the path using the electronic motion controller to actuate the plastic actuator coupled to the selectively controllable segment; and using the electronic motion controller to automatically deform the plastic actuator coupled to an automatically controllable segment to alter the geometry of the automatically controllable segment to assume the curve along the path.

In a further aspect of the invention, the plastic actuator is an electrorheological plastic actuator. In another aspect, the method includes advancing the instrument distally while automatically controlling the plastic actuators in the proximal automatically controllable segments to propagate the curve proximally. In another aspect, the method includes withdrawing the instrument proximally while automatically controlling the plastic actuators in the segments to propagate the curve distally along the instrument. In another aspect, the method includes measuring the advancing or the withdrawing using a transducer, an axial transducer, or other indicator of position. In another aspect, the geometry of the segments are controlled by the actuation of the plastic actuators so that the curve remains approximately fixed in space as the instrument is advanced proximally and/or withdrawn distally. In another aspect the path exists within an opening in a body. In another aspect, the path exists in an industrial space, such as a piping system. In another aspect, the path traverses a tube. In another aspect, the tube is an organ in a body. In another aspect, the instrument is an endoscope and the path is along a patient's colon.

In another aspect of the invention, there is provided an endoscope having a plurality of articulating segments wherein the shape of each segment is altered by the actuation of an electroactive polymer actuator operable in air. As used herein, “operable in air” refers to the nature of numerous activated polymers to be operable without reliance on an electrolyte or other transfer medium for function of the actuator. Operable in air refers to the lack of a requirement for such a medium for operation of the polymer actuator to proceed. Conductive polymer based actuators in particular are not operable in air because such polymers require immersion in or to be surrounded by an electrolyte for proper operation. “Operable in air” does not limit the environment where operation of non-electrolyte operating polymer actuators is possible.

In another aspect of the invention, the shape of each segment is altered by the cooperative actuation of two or more electroactive polymer actuators operable in air. In another aspect of the invention, at least one electroactive polymer actuator operable in air is inactive while at least one electroactive polymer actuator operable in air is actuated. In another aspect of the invention, the electroactive polymer actuator operable in air is actuated by Coulomb forces. In another aspect of the invention, the electroactive polymer actuator operable in air is actuated by a force selected from the group consisting of: electrostrictive, electrostatic, piezoelectric, and ferroelectric. In another aspect of the invention, the electroactive polymer actuator operable in air is categorized as an electronic electroactive polymer. In another aspect of the invention, each segment further comprises a plurality of electroactive polymer actuators operable in air, the plurality of electroactive polymer actuators configured such that the segment is capable of bending along an axis related to the longitudinal axis of the segment. In another aspect, the segment is capable of bending along at least two axes relative to the longitudinal axis of the segment.

In another aspect of the invention, there is provided an electronic motion controller configured to actuate the at least one electroactive polymer actuator in each articulating segment. In another aspect of the invention, the electroactive polymer actuators in a portion of the articulating segments are selectively controllable to follow a curve and the electroactive polymer actuators in another portion of the articulating segments are automatically controllable by the electronic motion controller to propagate the curve along the automatically controllable articulating segments while the endoscope advance through the curve. In another aspect of the invention, an electroactive polymer actuator is connected between two adjacent articulating segments such that actuation of the electroactive polymer actuator results in relative movement between the two adjacent articulating segments. In another aspect of the invention, the electroactive polymer actuator is a ring disposed about the circumference of an articulating segment. In another aspect of the invention, the electroactive polymer actuator is disposed about the periphery of the articulating segment. In another aspect of the invention, three electroactive polymer actuators are spaced about an articulating segment. In another aspect of the invention, the electroactive polymer actuators are uniformly spaced. In another aspect of the invention, expansion of the electroactive polymer in the electroactive polymer actuator bends the articulating segment. In another aspect of the invention, contraction of the electroactive polymer in the electroactive polymer actuator bends the articulating segment.

In another aspect of the invention, there is provided an endoscope having an elongate body, at least one electronic electroactive polymer actuator that when actuated bends at least a portion of the elongate body into a desired curve at a position; and an electronic motion controller configured to actuate the at least one electronic electroactive polymer actuator to bend at least a portion of the elongate body into the desired curve and to propagate the desired curve along the unbent portion of the elongate body as the unbent portion of the elongate body passes the position. In another aspect of the invention, the curve is a portion of a pathway. In another aspect of the invention, the pathway is a tubular pathway. In another aspect of the invention, the pathway is within a human body. In another aspect of the invention, the pathway is within a human colon. In another aspect of the invention, the elongate body comprises a plurality of segments. In another aspect of the invention, the at least one electronic electroactive polymer actuator bends at least a portion of the elongate body into a desired curve by causing relative movement between adjacent segments.

In another aspect of the invention, the at least one electronic electroactive polymer actuator is connected between two or more segments. In another aspect of the invention, the electronic electroactive polymer actuator is a sheet disposed about the elongate body, the sheet having a plurality of active areas and a plurality of inactive areas wherein the plurality of active areas are positioned to bend the elongate body. In another aspect of the invention, the electronic motion controller selectively actuates the active areas to propagate the desired curve along the elongate body. In another aspect of the invention, the elongate body is a continuous bendable structure. In another aspect of the invention, the at least one electronic electroactive polymer actuator is a rolled electroactive polymer actuator. In another aspect of the invention, the at least one electronic electroactive polymer actuator is a rolled electroactive polymer actuator.

In another aspect of the invention, there is provided an articulating instrument including at least two segments, each segment having an outer surface and an inner surface and comprising at least two internal actuator access ports disposed between the outer surface and the inner surface; and at least one electromechanical actuator extending through each of the internal actuator access ports and coupled to the at least two segments so that actuation of the at least one electromechanical actuator results in deflection between the at least two segments. In one aspect, the at least one electromechanical actuator, when activated by an electric field, demonstrates an induced strain proportional to the square of the electric field. In another aspect of the invention, the at least one electromechanical actuator is an actuated polymer actuator. In another aspect of the invention, the actuated polymer actuator operates without an electrolyte. In another aspect of the invention, the actuated polymer actuator activation mechanism utilizes coulomb forces. In another aspect of the invention, the actuated polymer actuator activation mechanism utilizes electrostrictive forces, electrostatic forces, piezoelectric forces or ferroelectric forces. In another aspect of the invention, the polymer actuator is a ferroelectric polymer. In another aspect of the invention, the polymer actuator comprises a polymer demonstrating piezoelectric behavior. In another aspect of the invention, the polymer actuator comprises an electret material. In another aspect of the invention, the polymer actuator is a dielectric electroactive polymer. In another aspect of the invention, the actuated polymer actuator activation mechanism comprises non-electrically activated the polymer actuator. In another aspect of the invention, the polymer actuator is a chemically activated polymer. In another aspect of the invention, the polymer actuator is a shape memory polymer. In another aspect of the invention, the polymer actuator is an McKibben artificial muscle. In another aspect of the invention, the polymer actuator is a light activated polymer. In another aspect of the invention, the polymer actuator is a magnetically activated polymer. In another aspect of the invention, the polymer actuator is a thermally activated polymer gel. In another aspect of the invention, the actuated polymer actuator activation mechanism utilizes electrochemical forces. In another aspect of the invention, the actuated polymer actuator activation mechanism utilizes ionic forces without a conductive polymer. In another aspect of the invention, the actuated polymer actuator activation mechanism utilizes ionic forces with a conductive polymer. In another aspect of the invention, a sheath extends between the at least two segments. In another aspect of the invention, the segments are continuous. In another aspect of the invention, the segments are annular. In another aspect of the invention, at least one of the access ports has a regular geometric shape. In another aspect of the invention, at least one of the access ports has a regular geometric shape selected from the group consisting of: circle, rectangle, oval; ellipse or polygonal. In another aspect of the invention, at least one of the access ports has a compound geometric shape. In another aspect of the invention, the sheath is attached to the outer surface of the at least two segments. In another aspect of the invention, the sheath is attached to the inner surface of the at least two segments. In another aspect of the invention, the sheath is attached to the inner surface of the at least two segments and another sheath is attached to the outer surface of the at least two segments.

In another aspect of the invention there is provided a segmented instrument including a plurality of segments; a sheath comprising a polymer layer and a pre-strained polymer layer having an active area, the sheath disposed about the plurality of segments wherein providing a voltage across a portion of the pre-strained polymer layer produces a deflection between at least two of the plurality of segments. In another aspect of the invention, the sheath is disposed about the plurality of segments so as encircle the plurality of segments. In another aspect of the invention, the sheath is disposed about the plurality of segments so as encircle the plurality of segments to form multiple layers of the sheath about the plurality of segments. In another aspect of the invention, the sheath is disposed about the plurality of segments to form a working channel defined by the plurality of segments and the sheath. In another aspect of the invention, the sheath is disposed about the plurality of segments on the outer perimeter of the plurality of segments. In another aspect of the invention, the sheath is disposed about the plurality of segments on the inner perimeter of the plurality of segments. In another aspect of the invention, the sheath comprises a compound laminate polymer actuator.

In another aspect of the invention, there is provided an articulating instrument, comprising an elongated, flexible, tubular body of multi-layered wall construction having a selectively steerable distal end for insertion into a body and an automatically controllable proximal end; at least one pair of structural elements within the flexible tubular body at axially spaced locations; at least one pair of compliant electrodes forming an active area on at least one polymer layer included in said multi-layered wall construction, the at least one pair of complaint electrodes between said at least one pair of structural elements; and control means for selectively activating the active area thereby making the portion of the elongated, flexible, tubular body between the at least one pair of structural elements selectively steerable or automatically controllable. In another aspect of the invention, the outermost layer of the multi-layered wall construction is the outer layer of the articulating instrument. In another aspect of the invention, an outer flexible sheath concentrically surrounds the flexible tubular body. In another aspect of the invention, at least one pair of compliant electrodes forming an active area on at least one polymer layer are part of an electrically activated polymer actuator. In another aspect of the invention, at least one pair of compliant electrodes forming an active area on at least one polymer layer are part of an ionically activated polymer actuator. In another aspect of the invention, at least one pair of compliant electrodes forming an active area on at least one polymer layer are part of a non-electrically activated polymer actuator. In another aspect of the invention, multi-layered wall construction includes a plastic actuator formed using a laminate polymer sheet structure. In another aspect of the invention, the laminate polymer sheet structure includes strained polymers and/or unstained polymers. In another aspect of the invention, the active area provides one planar direction of polymer deformation. In another aspect of the invention, the active area provides two planar directions of polymer deformation. In another aspect of the invention, the at least one pair of compliant electrodes comprises electrode patterning that produces multiple degrees of freedom of polymer deformation. In another aspect of the invention, an elongated, flexible, tubular body of multi-layered wall construction comprises a compound laminate polymer actuator.

In another aspect of the invention there is provided a bendable instrument, comprising an elongate body having a distal end and a proximal end, the elongate body having a pre-bias shape; and at least one activated polymer actuator coupled to the elongate body such that when activated the at least one activated polymer actuator alters at least a portion of the elongate body out of the pre-bias shape. In another aspect of the invention, the at least one activated polymer actuator comprises an electrically activated polymer actuator. In another aspect of the invention, the at least one activated polymer actuator comprises an ionically activated polymer actuator. In another aspect of the invention, the at least one activated polymer actuator comprises a non-electrically activated polymer actuator. In another aspect of the invention, the pre-bias shape is related to a typical pathway used in a surgical procedure. In another aspect of the invention, the pre-bias shape is related to a portion of the vasculature. In another aspect of the invention, the pre-bias shape is related to a portion of the skeleton. In another aspect of the invention, the pre-bias shape is related to the shape of an organ. In another aspect of the invention, the pre-bias shape is related to an internal shape of an organ. In another aspect of the invention, the pre-bias shape is related to the internal shape of a heart. In another aspect of the invention, the pre-bias shape is related to the internal shape of a colon. In another aspect of the invention, the pre-bias shape is related to the internal shape of the gut. In another aspect of the invention, the pre-bias shape is related to the internal shape of the throat. In another aspect of the invention, the pre-bias shape is related to an external shape of an organ. In another aspect of the invention, the pre-bias shape is related to the external shape of the heart. In another aspect of the invention, the pre-bias shape is related to the external shape of the liver. In another aspect of the invention, the pre-bias shape is related to the external shape of a kidney.

In another aspect of the invention, there is provided an articulating instrument, comprising an elongate body having a plurality of segments; a first portion of the plurality of segments forming a selectively steerable distal portion; a second portion of the plurality of segments forming an automatically controllable proximate portion; at least one activated polymer actuator that when actuated articulates or bends either the first or second portion of the plurality of segments; and an electronic motion controller configured to activate the at least one activated polymer actuator and to propagate a desired curve from the first portion to the second portion. In another aspect of the invention, the at least one activated polymer actuator actuates both the first and second portion. In another aspect of the invention, the at least one activated polymer actuator comprises a compliant electrode. In another aspect of the invention, the at least one activated polymer actuator comprises a charge distribution layer. In another aspect of the invention, the at least one activated polymer actuator comprises a compound laminate polymer actuator. In another aspect of the invention, the at least one activated polymer actuator comprises a rolled activated polymer actuator. In another aspect of the invention, the rolled activated polymer actuator is a compound rolled activated polymer actuator. In another aspect of the invention, the at least one activated polymer actuator comprises an ionically actuated polymer actuator that actuates without an electrolyte. In another aspect of the invention, the at least one activated polymer actuator comprises a conductive polymer and a compliant electrode. In another aspect of the invention, the at least one activated polymer actuator comprises a conductive polymer and a charge distribution layer. In another aspect of the invention, the at least one activated polymer actuator comprises a conductive polymer and a compound laminate polymer actuator. In another aspect of the invention, the at least one activated polymer actuator comprises an electrically activated polymer. In another aspect of the invention, the at least one activated polymer actuator comprises a non-electrically activated polymer.

### BRIEF DESCRIPTION OF THE DRAWINGS

FIGS. 1(a) to 1(c) show articulation of a portion of an endoscope using electro-polymeric materials when the material is contracted and/or expanded.

FIGS. 2(a) and 2(b) show perspective and end views, respectively, of a segment capable of bending along at least two axes.

FIGS. 2(c) and 2(d) show perspective and end views, respectively, of the segment bending in at least two directions.

FIGS. 2(e) and 2(f) illustrate an embodiment of an articulating instrument having a pre-set bias.

FIGS. 3(a) to 3(c) show end views of various possible configurations for positioning the electro-polymeric materials about a segment.

FIGS. 4(a) to 4(c) show articulation of a portion of an endoscope using electro-polymeric materials positioned between two adjacent segments.

FIG. 5(a) shows a perspective view of segments having electro-polymeric materials formed in a continuous band about the segments.

FIGS. 5(b) and 5(c) show end views of different configurations for positioning regions of electro-polymeric material about the segment circumference.

FIGS. 6(a) and 6(b) show side and cross-sectional end views, respectively, of a continuous band of electro-polymeric material extending over several segments or joints.

FIGS. 7(a) to 7(c) show articulation of a portion of an endoscope using electro-polymeric materials positioned over a length of flexible material.

FIG. 8(a) shows a perspective view of a flexible material having electro-polymeric materials formed in a continuous band about the material.

FIGS. 8(b) and 8(c) show end views of different configurations for positioning regions of electro-polymeric material about the circumference.

FIGS. 9(a) and 9(b) show side and cross-sectional end views, respectively, of a continuous band of electro-polymeric material extending over a length of the endoscope.

FIGS. 10(a) and 10(b) show side and end views, respectively, of a plurality of links connected together via hinges, joints, or universal joints.

FIGS. 10(c) and 10(d) show electro-polymeric material formed in individual lengths and in a continuous band, respectively, about a portion of the endoscope.

FIG. 10(e) shows a continuous sleeve of electro-polymeric material placed around the circumference of a number of segments.

FIG. 11 shows a length of electro-polymeric material having electrodes on either side to create a voltage potential through the electro-polymeric material.

FIG. 12 shows patterns for conductive ink that may be placed onto the electro-polymeric material that would allow for large degrees of stretching and contracting.

FIG. 13 shows a schematic illustration of individual conductors for connection to a controller using a separate wire or pair of wires.

FIG. 14 shows a schematic illustration of a network of small controllers that are each capable of switching and controlling a smaller number of electrodes for the electro-polymeric material.

FIGS. 15A and 15B illustrate a top view of a transducer portion before and after application of a voltage, respectively, in accordance with one embodiment of the present invention.

FIGS. 16A-16D illustrate a rolled electroactive polymer device in accordance with one embodiment of the present invention.

FIG. 16E illustrates an end piece for the rolled electroactive polymer device of FIG. 16A in accordance with one embodiment of the present invention.

FIG. 17A illustrates a monolithic transducer comprising a plurality of active areas on a single polymer in accordance with one embodiment of the present invention.

FIG. 17B illustrates a monolithic transducer comprising a plurality of active areas on a single polymer, before rolling, in accordance with one embodiment of the present invention.

FIG. 17C illustrates a rolled transducer that produces two-dimensional output in accordance with one environment of the present invention.

FIG. 17D illustrates the rolled transducer of FIG. 3C with actuation for one set of radially aligned active areas.

FIGS. 17E-G illustrate exemplary vertical cross-sectional views of a nested or compound rolled electroactive polymer device in accordance with one embodiment of the present invention.

FIGS. 17H-J illustrate exemplary vertical cross-sectional views of a nested or compound rolled electroactive polymer device in accordance with another embodiment of the present invention.

FIGS. 18A-18F illustrate alternative segment embodiments.

FIGS. 19A and 19B illustrate additional embodiments of activated polymer segments.

FIGS. 20A-20C illustrate articulating instrument embodiments actuated or manipulated using embodiments of rolled and compound rolled (nested) polymer actuators.

FIG. 21 illustrates another embodiment of a flexible member actuated by a number of active areas on a polymer sheet.

FIG. 22 illustrates another embodiment of a flexible member actuated by a number of active areas on a polymer sheet having integrated deflection measurement capability.

FIG. 23 illustrates another embodiment of a flexible member actuated by a number of active areas.

FIGS. 24 and 25 illustrate embodiments of compound laminate polymer actuators and multiple active areas.

FIG. 26 illustrates an embodiment of a hybrid articulating instrument.

FIGS. 27 and 28 illustrate an embodiment of the “follow the leader” technique applied to an exemplary articulating instrument.

FIGS. 29(a)-(d) illustrate an embodiment of a variable curvature segment.

FIGS. 30(a)-(e) illustrate an embodiment of variable curvature using non-activated electrodes.

### DETAILED DESCRIPTION OF THE INVENTION

A variety of electromechanical actuators based on the principal that certain types of polymers can change shape under certain conditions of stimulation have been under investigation for decades. During the 1990's, widespread international research was performed, numerous papers were published and several conferences held regarding activated polymer actuators. In January 2001, this research was organized by Yoseph Bar-Cohen in a book he edited entitled “Electroactive Polymer (EAP) Actuators as Artificial Muscles: Reality, Potential and Challenges” (SPIE Press, January 2001). As used herein, activated polymers refer generally to the families of polymers that exhibit change when subjected to an appropriate stimulus. See, for example, Bar-Cohen Topics 1, 3, and 7, Chapters 1 (pp. 1-38), 4 (pp. 89-117), 5 (pp. 123-134), 6 (pp. 139-184), 7 (pp. 193-214), 8 (223-243), and 16 (457-493) all of which are incorporated herein in their entirety.

One manner of categorizing activated polymers is by type of activation mechanism. Such categorization used by Bar-Cohen, and adopted herein, includes: non-electrically actuated polymers, ionically actuated polymers and electronically actuated polymers. There are numerous subcategories within each type of activation mechanism. Non-electrically activated polymers include chemically activated polymers, shape memory polymers, McKibben artificial muscles, light activated polymers, magnetically activated polymers, thermally activated polymer gels and polymers activated utilizing electrochemical action.

Ionically activated polymers include the groupings of electroactive polymer gels, ionomeric polymer-metal composites, conductive polymers, and carbon nanotubes. In one aspect, the invention provides an articulating instrument that is actuated or manipulated through the controlled use of an ionically activated polymer actuator activated without the use of an electrolyte. In a further aspect, the ionically activated polymer actuator comprises an electroactive polymer gel. In a further aspect, the ionically activated polymer gel actuator comprises a physical gel, a chemical gel, a chemically actuated gel, or an electrically actuated gel. In a further aspect, the ionically activated polymer actuator comprises an ionomeric polymer-metal composite. In a further aspect, the ionically activated polymer actuator comprises a carbon nanotube. In a further aspect, the ionically activated polymer actuator activates resulting in movement of the articulating instrument without the ionically activated polymer undergoing an oxidation/reduction process.

Electronically activated polymers include polymers activated using Coulomb forces, electrical forces, as well as electrostrictive, electrostatic, piezoelectric and/or ferroelectric forces. In a further aspect, the invention provides an articulating instrument that is actuated or manipulated through use of an electromechanical actuator from the category of an electronic electroactive polymer based actuator. In one aspect, an electronic electroactive polymer based actuator is used to articulate the controllable segments of an endoscope, including the distal steerable portion. In another aspect, embodiments of the electronic electroactive polymer based actuator include, but are not limited to, non-doped polymers, dielectric elastomers, electrostatically stricted polymers, electrostrictor polymer (i.e., polyvinylidene fluoride-triflouroethylene copolymer or P(VDF-TrFE)), polyurethane (such as manufactured by Deerfield: PT6100S), silicone (such as manufactured by Dow Corning: Sylgard 186), fluorosilicone (such as manufactured by Dow Corning: 730), fluoroelastomer (such as manufactured by LaurenL143HC), polybutadiene (such as manufactured by Aldrich: PBD), isoprene natural rubber latex, acrylic, acrylic elastomer, pre-strained dielectric elastomer, acrylic electroactive polymer artificial muscle, silicone (CF19-2186) electroactive polymer artificial muscle.

In another aspect, articulating instruments according to embodiments of the present invention employ a plastic actuator formed using a laminate polymer sheet structures including combinations of pre-strained polymers, unstrained polymers, compliant electrodes, active areas creating one planar direction of polymer deformation, active areas creating two planar directions of polymer deformation, compliant electrode patterning that produces multiple degrees of freedom and combinations of the above.

In some embodiments, an activated polymer is pre-strained. It is believed that the pre-strain improves conversion between electrical and mechanical energy. In one embodiment, pre-strain improves the dielectric strength of the polymer. The pre-strain allows the electroactive polymer to deflect more and provide greater mechanical work. Pre-strain of a polymer may be described in one or more directions as the change in dimension in that direction after pre-straining relative to the dimension in that direction before pre-straining. The pre-strain may comprise elastic deformation of a polymer and be formed, for example, by stretching the polymer in tension and fixing one or more of the edges while stretched. In one embodiment, the pre-strain is elastic. After actuation, an elastically pre-strained polymer could, in principle, be unfixed and return to its original state. The pre-strain may be imposed at the boundaries using a rigid frame or may be implemented locally for a portion of the polymer.

In one embodiment, pre-strain is applied uniformly over a portion of an active polymer to produce an isotropic pre-strained polymer. By way of example, an acrylic elastomeric polymer may be stretched by 200-400 percent in both planar directions. In another embodiment, pre-strain is applied unequally in different directions for a portion of the polymer to produce an anisotropic pre-strained polymer. In this case, the polymer may deflect greater in one direction than another when actuated. While not wishing to be bound by theory, it is believed that pre-straining a polymer in one direction may increase the stiffness of the polymer in the pre-strain direction. Correspondingly, the polymer is relatively stiffer in the high pre-strain direction and more compliant in the low pre-strain direction and, upon actuation, the majority of deflection occurs in the low pre-strain direction. By way of example, an acrylic elastomeric polymer used may be stretched by 100 percent in a first direction and by 500 percent in the direction perpendicular to the first direction. Additional details related to pre-straining activated polymers may be found in U.S. Pat. No. 6,664,718 to Pelrine et al. entitled “Monolithic Electroactive Polymers,” the entirety of which is incorporated herein by reference.

In other aspects of the invention, articulating instruments according to embodiments of the present invention utilize a plastic electromechanical actuator that relies on actuation from other materials, for example, infused mixtures of polymer gels with or without electrorheological fluid, electrorheological fluid, polydimethyl siloxane, polyacrylonitrile, carbon nanotubes and carbon single-wall nanotubes (SWNT).

Articulating instruments include a number of different types of articles including, for example, wireless endoscopes, robotic endoscopes, catheters, specific designed for use catheters such as, for example, thrombolysis catheters, electrophysiology catheters and guide catheters, cannulas, surgical instruments or introducer sheaths or other procedure specific articulating instruments.

Additionally, articulating instruments include steerable endoscopes, catheters and insertion devices for medical examination or treatment of internal body structures. Many such instruments are described in the following U.S. patents and U.S. patent applications, the disclosures of each are incorporated herein by reference in their entirety: U.S. Pat. Nos. 6,610,007; 6,468,203; 4,054,128; 4,543,090; 4,753,223; 4,873,965; 5,174,277; 5,337,732; 5,383,852; 5,487,757; 5,624,380; 5,662,587; 6,770,027; 6,679,836 and U.S. patent application Ser. No. 09/971,419 (notice of allowance Feb. 24, 2004, issue fee paid May 27, 2004).

A steerable, multi-segmented, computer-controlled endoscopic device is one specific example useful for discussion purposes to describe some of the embodiments of the present invention. Examples of such endoscopes are described in U.S. Pat. Nos. 6,468,203 and 6,610,007 both assigned to the Applicant. These steerable segmented endoscopes may be utilized for insertion into a patient's body, e.g., through the anus for colonoscopy examinations. An example of such a device and a method for advancement within a patient utilizing a serpentine “follow-the-leader” type motion may be seen in U.S. Pat. No. 6,468,203, which is co-owned and has been incorporated herein by reference above. Each of the segments of the endoscope may be individually actuated and controlled to create arbitrary shapes. Using such a “follow-the-leader” type algorithm, the device may be advanced into tortuous lumens or paths without disturbing adjacent tissue or objects.

Another variation on segment actuation for realizing the “follow-the-leader” motion is described in U.S. Pat. App. Serial No. 2002/0062062, filed Oct. 2, 2001. As described, one of the variations employs motors on board at least a majority of each individual segment. The motors described therein may be, in some embodiments of the present invention, replaced by electroactive polymer rotary clutch motors, such as those described in U.S. Patent Application Publication US 2002/0175598 to Heim et al. entitled, “Electroactive Polymer Rotary Clutch Motors,” or electroactive polymer rotary motors, such as those described in U.S. Patent Application Publication US 2002/0185937 to Heim et al. entitled, “Electroactive Polymer Rotary Motors,” both of which are incorporated herein by reference in their entirety. Adjacent segments may be pivoted relative to one another via hinges or joints. Another variation is described in U.S. Pat. App. Serial No. 2003/0045778, filed Aug. 27, 2002. As described, each of the segments of the multi-segmented endoscope may be actuated by push-pull cables or “tendons” (also known in the art as “Bowden cables”) connected to one or several actuators, e.g., motors, located remotely from the endoscopic device. Each of these publications is co-owned and incorporated herein by reference in its entirety.

As described herein, active polymer materials may be used in conjunction with multi-segmented articulating instruments to alter the relationship between, for example, two adjacent segments, a plurality of segments, a section of the articulating instrument or the entire length of the articulating instrument. Flexing of a portion of the instrument may result from inducing relative differences in size or length of material, e.g., active polymeric material, placed near, around or otherwise coupled to the instrument such that activation of the polymer results in controlled articulation of the instrument. For example, actuators utilizing an active polymer material may be located on opposing sides of a portion of an endoscope such that activation of the active polymer material results in the scope bending towards the side having the activated polymer actuator. In an alternative embodiment, another actuator utilizing an active polymer material may be located in opposition the earlier mentioned actuator so as to either not contract or to expand along the opposing side to facilitate bending or pivoting of that portion of the endoscope. The resulting shape will have the contracted portion of material along the inner radius, and the un-contracted or expanded length of material along the outer radius.

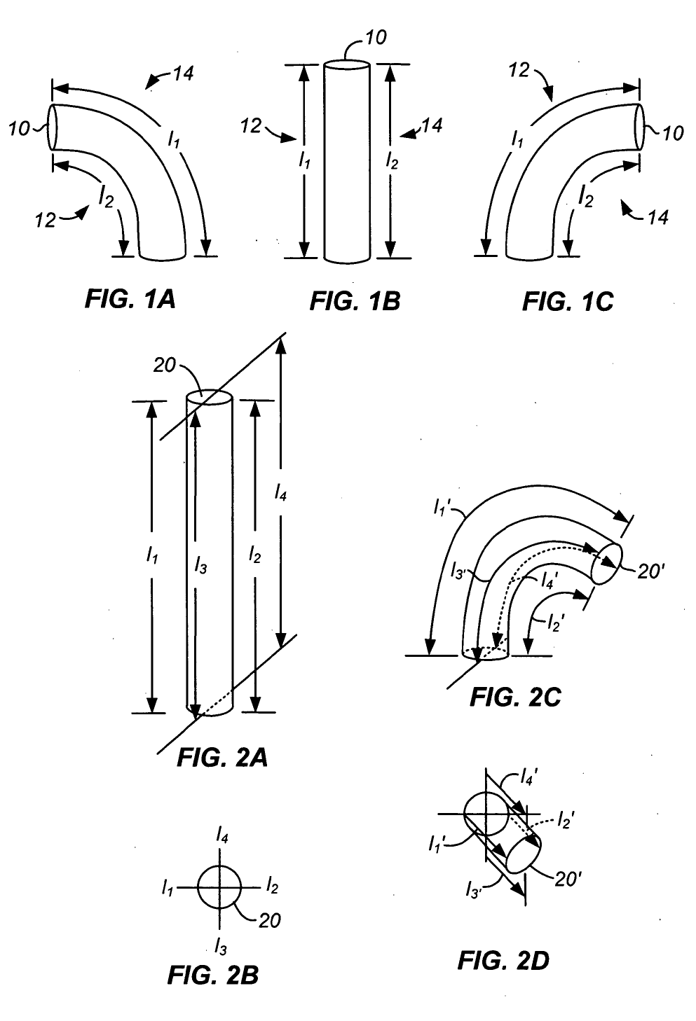

Consider a segment 10 having a first side 12 and a second side 14. Active polymer material or actuators are provided along the sides (not shown). When neither actuator or material is activated, the segment remains in a neutral position (FIG. 1b). On the other hand, FIG. 1(a) shows the case where material located along the length of a first side 12 of the segment 10 shown, L₁, is less than the length of material located along a second opposing side 14, L₂, and the resulting bending of the segment towards the first side 12. FIG. 1(b) shows the case where the length of the first side 12, L₁, is equal to the length of the second side 14, L₂, and the resulting straight, unbent, shape of the segment 10. FIG. 1(c) shows the case where the length of the first side 12, L₁, is greater than the length of the second side 14, L₂, and the resulting bending of the segment 10 towards the second side 14.

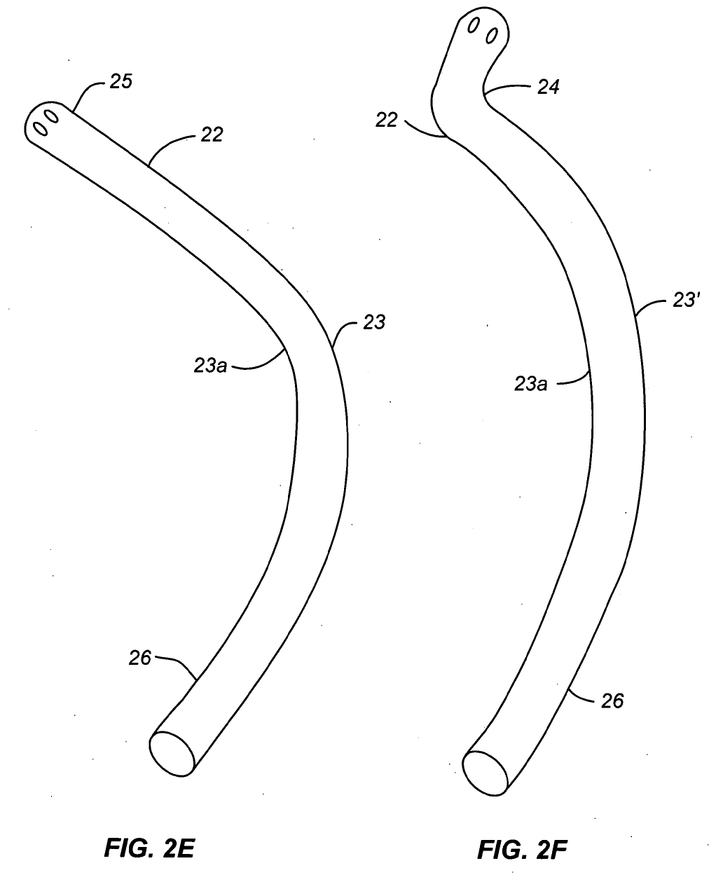

It is generally desirable to control the bending of the articulating instrument in all or as many directions as possible as suits the application. In one preferred embodiment, active polymer based actuators provide control rendering a segment capable of bending along at least two axes relative to a segment longitudinal axis. Segment 20 illustrates one configuration to achieve such control and articulation capable of bending along two axes (FIG. 2a-2d). FIGS. 2(a) and 2(b) illustrate side and top views, respectively, of segment 20. The segment 20 is straight, and the lengths of the sides L₁, L₂, L₃ and L₄ are all equal. FIGS. 2(c) and 2(d) illustrate side and top views, respectively, of an actuated or bent segment 20 or a segment 20.′ As a result of the controlled actuation of activated polymer actuators coupled to the segment 20,′ the segment 20′ has been articulated in two directions: towards the side denoted by L₂, and also out of the plane of the page towards the side denoted by L₃. In order to cause the depicted segment 20′ to bend as shown, length L₂′ may be made shorter than length L₁′, and length L₃′ may be made shorter than length L₄′, e.g. by causing the activated polymer materials or actuators located along L₂′ and L₃′ to contract. In this way, the segment 20′ may be caused to articulate, or bend, in two independent axes. Alternatively, the electro-polymeric materials along L₂′ and L₃′ may be remain un-actuated and the material along opposing sides L₁′ and L₄′ may be expanded to cause the resulting bending. In another alternative, all sides of the segment 20′ may be utilized in conjunction with another. For example, the material along sides L₂′ and L₃′ may be contracted while the material along sides L₁′ and L₄′ may be expanded simultaneously.

In yet another alternative, segment 20′ may represent an initial inactivated state for the segment that is pre-strained or has a bias condition with a predetermined and desired shape or curve. In this illustrative example, the segment 20′ is curved to the right in an inactivated state (FIGS. 2c and 2d). When the activated polymers or actuators coupled to the segment 20′ are activated, the segment is actuated into a straight condition. Pre-bias of a segment allows for actuation with fewer actuators. In this illustrative example, the actuator along side 12 may be removed since the pre-bias provides the curvature provided by the actuator in this position. During operation, the pre-bias is either reduced (i.e., less of a right turn), eliminated (i.e., straight up as in FIG. 2a) or articulated into another configuration as desired.

The use of pre-bias is also illustrated with articulating instrument 22 (FIG. 2e, 20. Articulating instrument 23 includes a plurality of segments (not shown for clarity) with selectively steerable distal portion 25 and an automatically controlled proximal portion 26. The articulating instrument 22 may be pre-biased into any desired curve. The curve may represent a typical pathway used, for example, in a surgical procedure such as an operation within the thoracic cavity, where the pre-bias shape is related to the likely shape of instrument when finally in position. The general pre-bias shape may be manipulated to fine tune the shape to patient specific anatomy. In another example, the pre-bias shape may relate to the pathway formed by vasculature or relate to the anatomy within an organ, such as the heart.

Articulating instrument 22 will now be described in relation to a use as a controllable, segmented colonoscope actuated through the use of active polymer layers or actuators. Once the articulating instrument 22 has been lubricated and inserted into the patient's colon through the anus A, the distal end is advanced through the rectum until the first turn in the colon is reached. This first turn is illustrated in FIG. 2f with bend 24. To negotiate the turn, the selectively steerable distal portion 25 is manually steered toward the sigmoid colon by the user through a steering control. The control signals from the steering control to the selectively steerable distal portion 25 are monitored by an electronic motion controller. Once the correct curve of the selectively steerable distal portion 25 for advancing the distal end of the instrument 22 into the sigmoid colon has been selected, the curve is logged into the memory of the electronic motion controller as a reference. Whether operated in manual mode or automatic mode, once the desired curve (24) has been selected with the selectively steerable distal portion 25, as the articulating instrument 22 advances distally, the selected curve 24 is propagated proximally along the automatically controlled proximal portion 26 using an electronic motion controller. As is common in “follow the leader” techniques (described below) the curve 24 remains fixed in space while the articulating instrument 22 advances distally through the sigmoid colon.

However, beyond the first turns to reach the sigmoid colon, traversing the colon may be thought of as a series of “left hand turns.” Consider, for example, that traversing the colon from the sigmoid colon into the descending colon, the descending colon into the transverse colon, and the transverse colon through the right (heptic) flexture into the ascending colon includes a series of left turns. As such, the pre-bias bend 23 is an example of a left hand pre-bias that may be used to approximate the general orientation of the articulating instrument once the colon has been traversed. In this way, in order for the instrument 22 to traverse the colon the pre-bias is selectively removed as it progresses. The pre-bias may also be removed selectively to more closely approximate the patient's anatomy. In alternative embodiments, the pre-bias may be shaped to any position other than the final position as described above.

FIG. 2f also illustrates how the instrument may be actuated in some portions while retaining the pre-bias condition in others. For example, the selectively steerable end 25 is articulated to form bend 24, the mid-region is actuated to diminish the pre-bias curvature while the proximal end retains the original pr-bias curvature. It is to be appreciated that the use of pre-bias may allow for fewer actuators to be needed to maintain the instrument in the final position or fewer actuators may be used overall. For example, in the left hand bias of instrument 22, actuators along the side 23a may be fewer or non-existent. Such an embodiment of the instrument 22 would thus be actuated through use of actuators to reduce, nullify or overcome and redirect the instrument out of the pre-bias shape.

There is provided a bendable instrument 22 having an elongate body with a distal end 25 and a proximal end 26. The elongate body is provided with a pre-bias shape. There is least one activated polymer actuator coupled to the elongate body such that when activated the at least one activated polymer actuator alters at least a portion of the elongate body out of the pre-bias shape. In one embodiment, the at least one activated polymer actuator comprises an electrically activated polymer actuator. In another embodiment, the at least one activated polymer actuator comprises an ionically activated polymer actuator. In yet another embodiment, the at least one activated polymer actuator comprises a non-electrically activated polymer actuator. In addition to or in combination with the pre-bias shapes described above, pre-bias shape embodiments also include: a pre-bias shape is related to: a typical pathway used in a surgical procedure, a portion of the vasculature; a portion of the skeleton, the shape of an organ, including both internal and external organ shapes. In some embodiments, the pre-bias shape is related to the internal shape of a portion of a heart, a colon, a gut, or a throat. In some embodiments, the pre-bias shape is related to the external shape of a portion of a heart, a liver, or a kidney.

In some embodiments, an articulating instrument is a restoring force that biases the entire assembly toward a substantially linear configuration in one embodiment, or into non-linear configurations or specialized configurations as described above. As discussed above, actuators may be used to deviate from this substantially linear configuration. It is to be appreciated that any of a number of conventional, known mechanisms can be provided to impart a suitable bias to the articulating instrument. For example, and as previously illustrated, an instrument may be disposed within an elastic sleeve, which tends to restore the system into a configuration determined by the strained, unstained or otherwise configured shape of the sleeve. Alternatively, springs or other suitably elastic members can be disposed in relation to structural elements of a segment to restore the instrument to a desired configuration, linear, non-linear or other shape as discussed elsewhere. In yet another alternative, the structural elements of the instrument itself may, alone or in combination with other suitable elastic or restorative members to maintain or restore the instrument to a desired configuration.

In some embodiments of the articulating instruments of the present invention, at least two controllable lengths of the sides of an instrument segment are desirable. In some embodiments, at least two controllable segment lengths would be needed to provide two independent axes in order to allow the segment to bend in any number of directions. In some embodiments, each of the sides or controllable lengths are independently actuatable. Alternatively, a single controllable length may be utilized for each axis, along with a biased spring-type element positioned to oppose the controllable length or actuator. In one alternative embodiment, fixed the lengths on the sides, of one axis and then vary the length of the opposing sides. With reference to FIG. 2(a), for example, if lengths L₁ and L₃ were fixed, then actuating the lengths L₂ and L₄ would enable the segment 20′ to bend in a number of directions.

In another alternative embodiment, three independently controllable actuators or activated polymer material may be coupled to the sides of an instrument to control the actuation of the instrument. Instead of being spaced at 90 degree intervals, as is shown in FIG. 2, the independently controllable actuators or activated polymer material could be spaced at 120 degree intervals or form 60 degree arc segments about the circumference of the articulating instrument. By extension, any number of controllable actuators or activated polymer material formed into sections (including longitudinal, horizontal or lateral sections) may be coupled to the articulating instrument or it's segments, or groups of segments to provide bending and/or articulation of the instrument as desired.

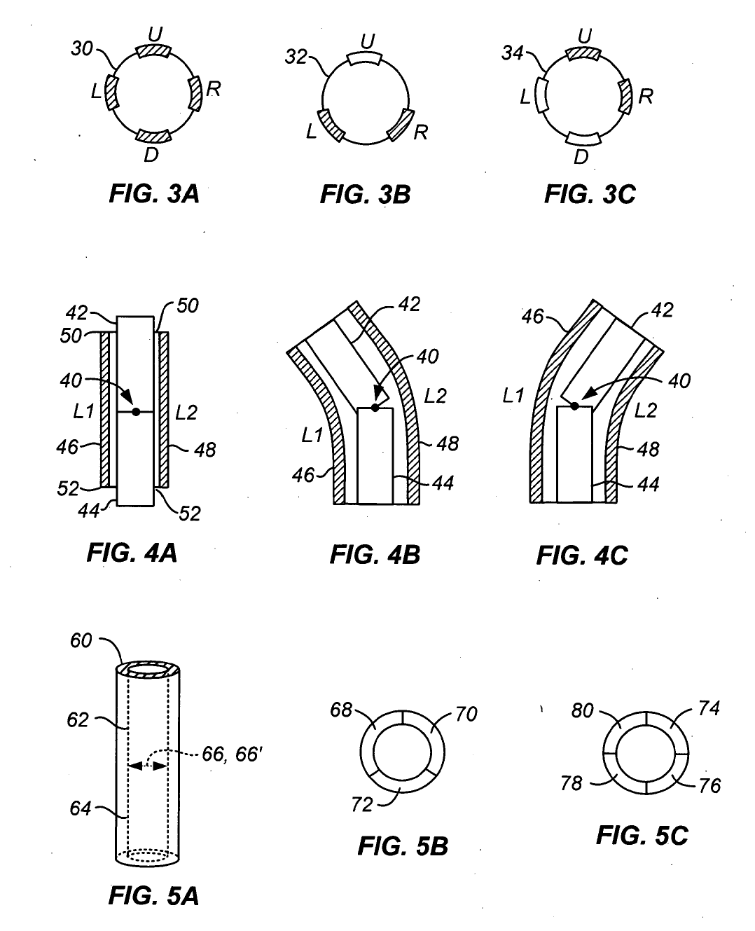

In some embodiments, it is preferable to control at least one pair of activated polymer actuators coupled to opposing sides of an instrument. This may result in four independently controllable sides or portions of a segment which may be utilized to determine the bending of the segment. This may facilitate the simplicity of computation for determining the desired or necessary bending. This may further result in desirable controllability and responsiveness when causing a segment to bend. For example, FIG. 3(a) shows a top view of a segment 30 in a configuration utilizing four independently controllable actuators along the sides for determining the length of the sides or bending of the segment 30. In this embodiment, the actuators (U, D, L, and R) are arranged on opposing sides about a circumference of the segment 30 at 90 degree intervals. Alternatively, segment 32 in FIG. 3(b) illustrates three independently controllable actuators along the sides (U, L, R) for determining the length of the sides. The three actuators U, L, R are spaced about the circumference of the segment 32 at 120 degree intervals. FIG. 3(c) shows yet another variation 34 showing two independently controllable sides U, R for determining the length of the sides of a segment 34 and two fixed-length sides D, L opposite with respect to sides U, R, arranged at 90 degree intervals.

Although the examples shown above are directed towards specific variations for placement of activated polymer materials and actuators circumferentially about a segment, these examples are intended to be illustrative and other variations and configurations for their placement are included within the scope of this disclosure.

In some embodiments, activated polymer materials and/or activated polymer based actuators may be configured for controlling the length of the sides of portions, or segments, of an articulated instrument to bend or otherwise manipulate the instrument into a desired direction, orientation or configuration. By positioning individually controllable pieces or regions of activated polymer material or actuators such that they may act on the segments of an instrument to modify, shorten, lengthen or otherwise alter the relative positions of segments or portions of the instrument and then controlling the contraction and/or activation of the activated polymers, the articulating instrument segments may be made to bend and flex as desired.

In one embodiment, pieces or lengths of activated polymer materials and/or activated polymer based actuators may be arranged around the periphery or circumference of a hinge or joint 40 between two adjacent segments 42, 44 (FIGS. 4(a) to 4(c)). The ends of the pieces 50, 52 of activated polymer materials and/or activated polymer based actuators 46, 48 may be fixed to the adjacent segments 42, 44 around the hinge or joint 40. As such, activation of or changes of length of the activated polymer materials and/or activated polymer based actuators 46, 48 will exert forces on the hinge or joint 40 and bend it in its axis of motion. As shown in FIG. 4(a), constriction of the length of active polymer material 46 on a first side L₁ is controlled so that it is the same length as that of the material 48 on a second side L₂, the hinge 40 will not be caused to bend, and will configure into a straight configuration. In this case, the hinge 40 may optionally be under equal tension from both activated polymer materials and/or activated polymer based actuators 46, 48, or it may be under no tension from either length L₁ or L₂.

To bend the joint or hinge to a first side towards L₁, as shown in FIG. 4(b), the length of polymeric material 46 may be caused to contract while the length L₂ of polymeric material 48 may be caused to relax or expand. To bend the joint or hinge 40 to the opposing second side towards L₂, as shown in FIG. 4(c), the length L₂ of polymeric material 48 may be caused to contract while the length L₁ of polymeric material 46 may be caused to relax or expand. The polymeric material may also be located inside an interstitial space′ pace or lumen defined within the adjacent segments 42, 44 and hinges 40. FIG. 4 is an exemplary embodiment where activated polymer materials and/or activated polymer based actuators are configured around the outside of the segments and hinges. Alternative configurations are also possible, such as a configuration where the activated polymer materials and/or activated polymer based actuators are disposed within or between the segments and/or hinges.

While the embodiment illustrated in FIG. 4 includes activated polymer actuators of equal lengths or sizes (i.e., L₁ being equal in length to L₂), other embodiments of the invention are not, so limited. Other variations may utilize lengths, sizes and shapes of activated polymer actuators and/or material having different lengths about the same joint or hinge. In one embodiment, a first length L₁ may be longer or shorter than a second length L₂ when both lengths are in a neutral or non-activated configuration. When either or both lengths are stimulated to either contract or expand, the adjacent segments may be configured to bend at various angles about the joint or hinge relative to one another. Alternatively, activated polymer actuators and/or material of different lengths may be configured to effect a uniform bending of the segment about the longitudinal axis of the segment.

In another alternative embodiment, the design of the articulating instrument may be extended to two axes of bending by using a universal joint instead of a hinge. A universal joint allows for bending in any direction relative to the segment longitudinal axis. In this case, lengths of activated polymer material and/or activated polymer actuators may be arranged around the circumference of the segment across the universal joint such that adjacent segments may be caused to bend in any desired direction. This preferably utilizes at least two lengths of material arranged between the segments such that they are each able to effect motion of the joint in each of the two independent axes. In one embodiment, the minimum number of lengths of material or actuators is two. In other embodiments, any number may be used to cause the desired bending of the universal joint. In another specific embodiment, four lengths of activated polymer material or actuators are arranged in intervals around the periphery of the universal joint such that, when activated, they generate push and/or pull forces in each of the two independent axes of bending. In one embodiment, the interval is 90 degrees. In alternative embodiments, the interval is not a 90 degree interval but instead is in another arrangement suited to the particular geometry of the joint used.

Turning now to FIGS. 5a, b and c, there is illustrated another embodiment of an activated polymer actuated instrument of the present invention. In this embodiment, a continuous band of activated polymer material is formed into an annular ring 60 having a length and placed about two adjacent segments 62, 64. A hinge 66 is positioned between the segments 62, 64. The activated polymer ring 60 is disposed about the periphery of a hinge 66 that may bend in one or more axes. Alternatively, the segments 62, 64 may be coupled together using a universal joint 66′ that may bend in two or more axes, as shown in FIG. 5(a). The annular ring 60 may be a single sheet of activated polymer material (FIG. 5a) having multiple active areas that deflect selected portions of the polymer to result in controllable movement of the segments 62, 64. In an alternative configuration, the annular ring may not be a single piece but instead a plurality of longitudinal activated polymer strips, such as polymer strips 68, 70 and 72 in FIG. 5b. In one embodiment, controllable activated polymer regions 68, 70, 72 individually (or alternatively, as a subset of the single piece, annular ring 60) are configured and controlled such that they may contract, relax, and/or expand as desired through the use of electrodes that may be energized, de-energized, and/or energized with polarities reversed to impart the desired shape or orientation of segments 62, 64. In one preferred embodiment, each of the controllable regions 68, 70, 72 or the single ring 60 are independently controlled. As such, a single piece or length of activated polymer material may be used to actuate either a hinge 66 or a universal joint 66′ in any desired direction.

While illustrated with three, any number of individually controllable regions of electro-polymeric material may be created. In some embodiments, the number of regions is greater than or equal to two. In one embodiment, the regions are arranged such that they act in the plane of the axis they control. For instance, three regions 68, 70, 72, as shown in FIG. 5(b) or four regions 74, 76, 78, 80, as shown in FIG. 5(c), may be utilized to individually control regions as desired to create the push and/or pull forces.

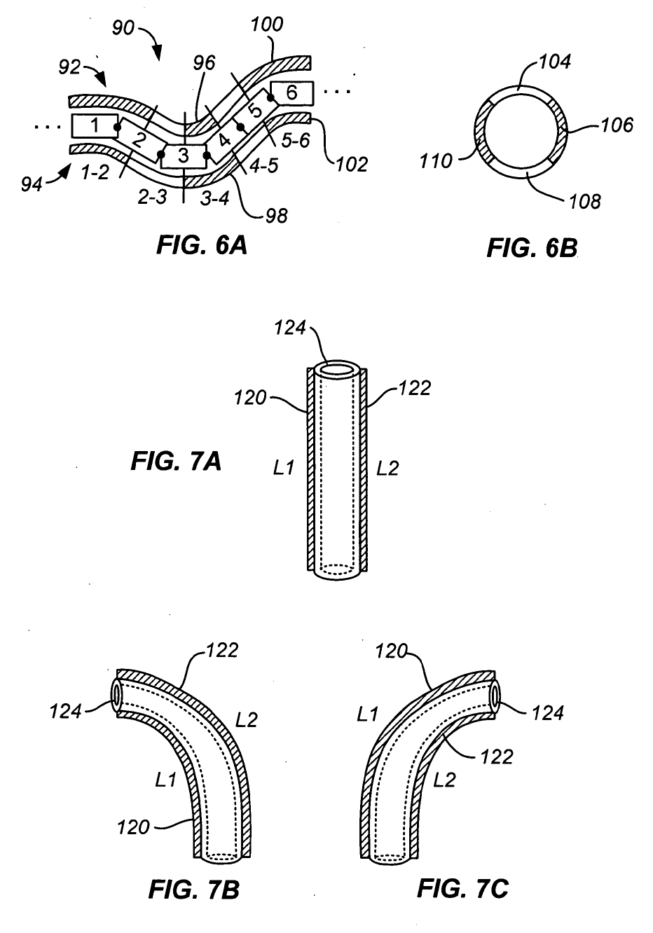

In yet another variation, a continuous band of electro-polymeric material that is formed in an annular ring and placed around the periphery of a segment may be made to be longer in length so that it extends over several, i.e., over at least two, hinges or universal joints, as shown in FIG. 6(a). It may be made in a single continuous piece and may be made to cover a portion of the length or even the entire length of the flexible endoscope structure. In this configuration 90, independently controllable regions of the electro-polymeric material, e.g., regions 96; 98, 100, 102 and so on, may be created and located so that they are able to exert bending forces on each hinge, joint, or universal joint along the length of the endoscope, or as many hinges, joints or universal joints as are contained within the sleeve of electro-polymeric materials 92, 94. The electro-polymeric material may be fixed to the hinged or jointed structure at or near the midpoint of rigid sections between the hinges or joints in order to impart force to the hinges and joints to make them bend, or optionally the electro-polymeric material may be unattached to the structure, and either impart forces to the structure using frictional contact and elasticity or cause the structure to conform to the shape it is controlled to take on with the electrodes. Alternately, the length of electro-polymeric materials may be located inside the segments, hinges and/or universal joints, in any interstitial space defined within.

In another embodiment, an multi-segment articulating instrument 90 includes a plurality of individually controllable regions (FIG. 6a). In this embodiment, the articulating instrument 90 includes 6 hinged segments covered by activated polymer material 92, 94. In one embodiment, the activated polymer material is divided into a plurality of controllable segments that correspond to the hinged portions between segments. When activated, these activated polymer materials produce controlled movement between segments about the hinge (i.e., segment 5-6 may be altered by controllable segment 100 or controllable segment section 102. Articulating instrument 90 may bend each hinge or joint in the desired directions through activation of the activated polymers in the individually controllable regions 96, 98, 100, 102 of polymer material 92, 94. In one embodiment of the articulating instrument 90, a continuous band of active polymer material that runs the length, or a subset of the length, of the instrument and forms a sheath. This sheath may be made of or coated by biocompatible materials, such as silicone, urethane, or any other biocompatible material as is commonly used in endoscopes or other medical devices, so that it may come in contact with living tissue without causing harm or damage. In one embodiment, the electrodes used to control the shape and length of the active polymer material or actuators are insulated or covered to prevent electric shock, which may also be accomplished with biocompatible materials. In another embodiment, the electrodes are compliant electrodes. In yet another embodiment, the sheath is part of a multi-layer laminate polymer actuator. In one embodiment, the sheath forms a disposable cover over a segmented structure comprising hinges and activated polymer materials coupled to the hinges. In another embodiment, the sheath is cleanable, washable and/or reusable.

FIG. 6(b) shows a cross-sectional view of an alternative embodiment of a controllable region. Rather than have the entire sleeve of activated polymer material, there may be provided sections of activated polymer material and non-activated polymer material. For example, sections 104, 110 may be the portions having activated polymers (for example, compliant electrodes distributed across a portion of their surface) while the sections 106, 108 would not have activated polymers or be formed from non-activated polymer material. Alternatively, each of the portions 104, 106, 108, 110 may be made of activated polymer materials and may each be controllable independently from one another. The sections need not be limited to the longitudinal sections illustrated. Other alternative embodiments include: more than four sections, a plurality of concentric longitudinal sections, annular sections, a plurality of concentric annular sections and combinations of longitudinal sections, annular sections and concentric sections.

In other alternative embodiments, a bendable instrument or articulating instrument does not use segments as in FIG. 6 but rather a continuous flexible material. As illustrated in FIG. 7, a representative segment 124 is made of a flexible material, such as a hose, tube, spring or any other continuous material that may be bent or flexed. In the illustrated embodiment, sections, pieces or lengths of activated polymer material 120, 122 is arranged around the periphery of the segment 124. The pieces of activated polymer material are coupled to the segment 124 such that activation of the polymer resulting in the desired deflection, bending or other actuation of the segment 124. The activated polymer material may be coupled to the structure of the segment 124 in any number of positions, for example, along the outside of the segment, the inside of the segment, only at the segment ends, continuously along the segment length, or in any other manner such that activation of the activated polymer material results in controlled changes in the shape, orientation, bending or overall geometry of the segment 124.

An exemplary actuation of segment 124 will now be described with reference to FIGS. 7a, 7b and 7c. As shown in FIG. 7(a), when the length of electro-polymeric material 120 on the first side with length L₁ is controlled so that it is the same length as that of the material 122 on the second side with length L₂, segment 124 will not be caused to bend, and will be in a straight configuration. In this case, the segment 124 may optionally be under equal tension from both activated polymer materials 120, 122, or, alternatively, the segment 124 be under no tension from either activated polymer. To bend the segment 124 to a first side, as shown in FIG. 7(b), the activated polymer material or actuator 120 on the left of segment 124 (L₁) may be caused to contract while the activated polymer material or actuator 122 on the right (L₂) is caused to relax or expand. To bend segment 124 to the right, as shown in FIG. 7(c), the activated polymer material or actuator 122 to the right of segment 124 (L₂) may be caused to contract while the activated polymer material or actuator 120 to the left (L₁) is caused to relax or expand. FIG. 7 shows the hose, tube or spring bending in one axis (left-right) for illustrative purposes, and may be extended to two axes and three dimensions by adding additional, individually controllable lengths of electro-polymeric material to cause the hose, tube or spring to bend in a plane out of the page (up-down).

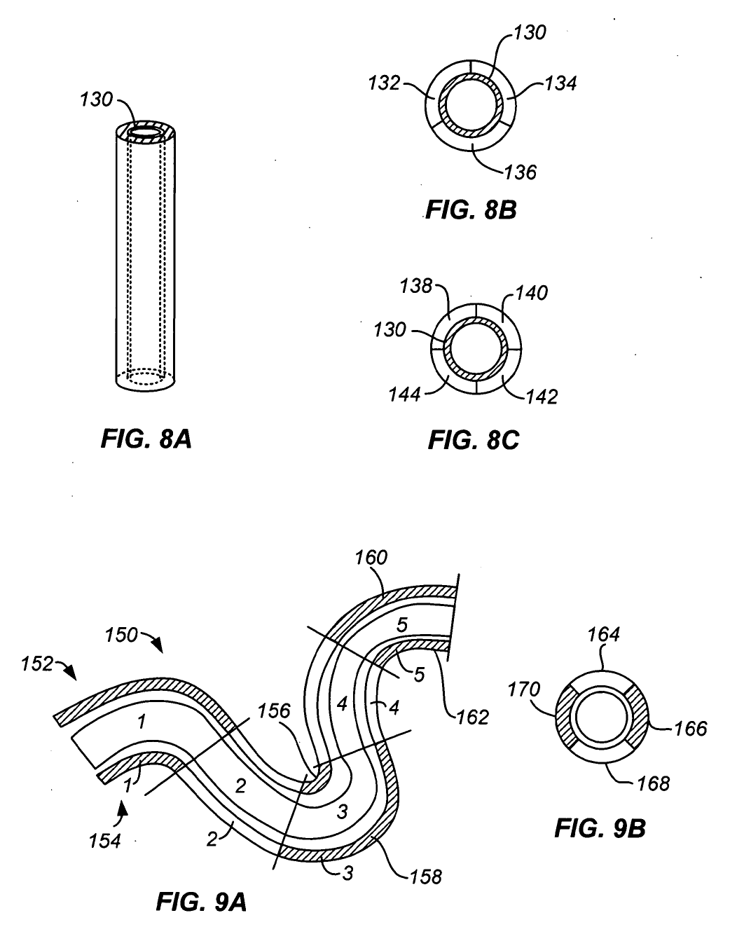

In yet another variation, a continuous band of activated polymer material may be formed in an annular ring and placed around the periphery of a segment 130, e.g., hose, tube, spring or any other continuous material that may be bent or flexed in any direction. In this configuration, as shown in FIG. 8(a), independently controllable regions 132, 134, 136 of activated polymer material are created such that they may contract, relax, and expand as desired through the use of electrodes that may be energized, de-energized, or energized with polarities reversed. In this way, a single piece of activated polymer material may be used to actuate a length of segment 130. Any number of individually controllable regions 132, 134, 136 of activated polymer material may be created. In one embodiment, there are two controllable regions. In another embodiment, there are three controllable regions as in the three regions 132, 134, 136 shown in FIG. 8(b). In yet another embodiment there are four or more controllable regions such as the four regions 138, 140, 142, 144 shown in FIG. 8(c). In any of the above described regions, the regions may be arranged such that they expand and/or contract in the plane of the axis they control and/or may be used to individually control regions to create push and/or pull forces on the segment 130.

FIG. 9(a) illustrates alternative embodiment of an articulated instrument of the present invention. Articulating instrument 150 includes in a continuous band of activated polymer material 152, 154 that is formed, in this embodiment, as an annular ring and may be placed around the periphery of or along the inner diameter of the interstitial space defined by a length of hose, tube, spring or any other continuous material 153 that may be bent or flexed in a desired direction. In some embodiments, the activated polymer material is of sufficient length such that it extends over several “segments.” In FIG. 9(a), five “segments” of the continuous structure are created because of the individual control over each of the controllable sections or regions 156, 158, 160, 162. These segments are defined as independently controllable sections that may be caused to bend in any direction. Segments may be chosen to be any desired length. In an exemplary embodiment where the articulating instrument is an endoscope the segments may, for example, range in length from, e.g., 1 cm to 10 cm. For other applications even smaller segment lengths may be used and will depend on the application. In some embodiments where the articulating instrument is intended to navigate the vasculature or other confined pathways, the segment length may be less than one cm, such as 50 mm or 25 mm.

The activated polymer material 152, 154 used may be made in a single continuous piece, and may be made to cover the entire length of the hose, tube, spring, or other flexible material making up the flexible endoscope structure 150. In this configuration, independently controllable regions 156, 158, 160, 162 of the activated polymer material are created and located so that they are able to exert bending forces on each segment along the length of the endoscope, or as many segments as are contained within the sleeve of the activated polymer material, which may be less than the entire length of the endoscope. The activated polymer material 152, 154 may be fixed to the hose, tube, spring, or other flexible material making up the endoscope at or near the endpoints of each of the segments in order to impart force to the segments to make them bend, or optionally the activated polymer material 152, 154 may be unattached to the structure, and either impart forces to the structure using frictional contact and elasticity or cause the structure to conform to the shape it is controlled to take on with the electrodes.

FIG. 9(a) illustrates an embodiment having individually controllable regions 156, 158, 160, 162 of activated polymer material configured to act such that they are able to bend each hinge or joint in the desired directions. In this structure, the continuous band of activated polymer material that runs the length, or a subset of the length, of the endoscope made of a series of segments forms a sheath. This sheath may be made of or coated by biocompatible materials, such as silicone, urethane, or any other biocompatible material as is commonly used in endoscopes or other medical devices, so that it may come in contact with living tissue without causing harm or damage. The electrodes used to control the shape and length of activated polymer material may be compliant electrodes and may also be insulated or covered to prevent electric shock, which may also be accomplished with biocompatible materials. In one embodiment, the sheath is disposable. In another embodiment, the sheath is cleanable and reusable.

FIG. 9(b) illustrates a cross-sectional view of one embodiment of one portion of the controllable region. Controllable region portions 166, 168 may be configured with the activated polymer material while portions 164, 170 may be made of non-activated polymer material. In another alternative embodiment, each of the controllable region portions 164, 166, 168, 170 may include activated polymer material and may each be controllable independently one from the others.

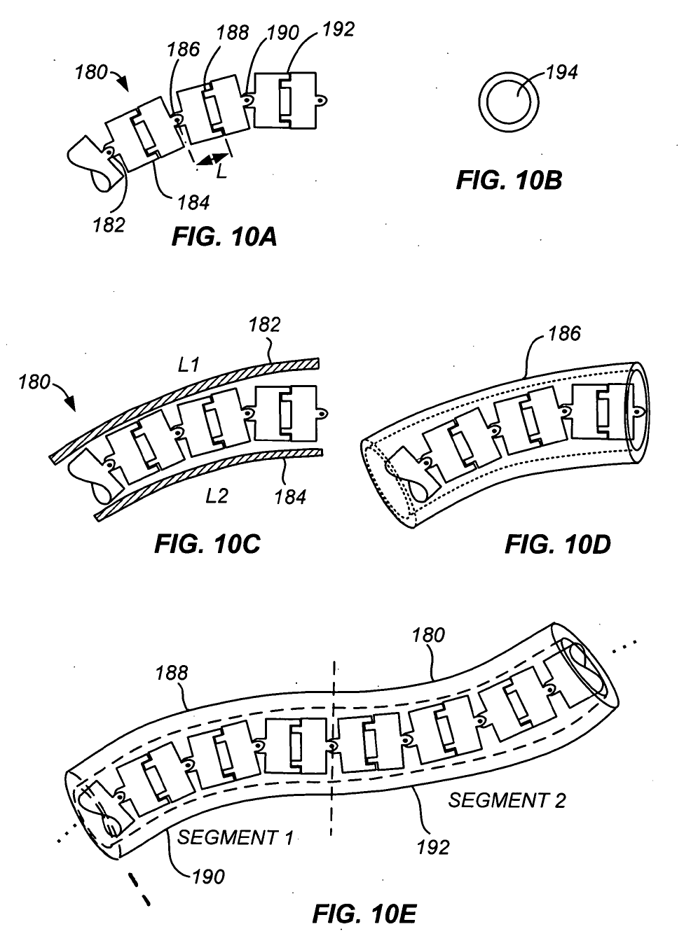

In yet another variation, a length 180 of hose, tube, spring, or alternate flexible material or structure may be comprised of a plurality of hinges, joints, or universal joints 182 to 192, as shown in FIG. 10(a). The hinges, joints, or universal joints 182 to 192 may be connected together to form a segment 180, shown in FIG. 10(a), which may then be caused to bend in two axes, e.g., via the use of activated polymer material. The hinges, joints, or universal joints 182 to 192 may define an inner lumen 194, or working channel, as shown in the end view of segment 180 in FIG. 10(b), which is large enough so that components may be assembled or passed within the defined lumen 194. Tools and components such as cables, tubes, working channels, optical fibers, and other tools, illumination bundles, etc., may be passed through the lumen 194. For arrangements that make use of hinges or joints that are configured to bend only in one axis (as opposed to universal joints, which are able to bend in at least two axes), it is preferable to alternate the orientation of the hinges or joints so that every other hinge or joint bends in one axis (e.g., left-right) with intermediate hinges or joints bending in another axis (e.g., transverse or up-down).

The spacing between the joints 182 to 192 lengthwise down the segment 180 is preferably small relative to the diameter of each link (e.g., 1:1 or less), so that the lengths of straight, un-articulated material covering the joint between adjacent links is correspondingly small. In this way, the series of discrete hinges, joints, or universal joints 182 to 192 may approximate the continuous shape of a flexible material (e.g., a hose, tube, spring, etc.). In this variation, activated polymer material may be used in any of the variations described above.

In one embodiment, illustrated in FIG. 10(c), individual pieces or lengths of activated polymer material 182, 184 may be used either outside the segments or inside to apply bending forces to the segments made of hinges or joints. Alternatively, as shown in FIG. 10(d), a continuous band 186 may be placed around the circumference of a segment or within the inner diameter of the segment that is the length of the segment or at least a partial length of the segment and is attached to the segment at or near the endpoints. In another alternative, as shown in FIG. 10(e), a continuous sleeve 188 may be placed around the circumference of a number of segments 190, 192 that may comprise the entire endoscope or a subset of the segments making up the endoscope. In the variations where a continuous band or sleeve is used, it may be preferable to configure the activated polymer material so that it has, in some embodiments, four individually controllable regions about the circumference per segment, and that these regions may exert push and/or pull forces in line with the axis of bending of the hinges or joints. Individually controllable pieces or lengths of activated polymer material, or individually controllable electrodes covering individual regions of activated polymer material, may be used to bend each of the segments individually in any desired direction. In addition, a sheath may be provided that is made of or coated by biocompatible materials, such as silicone, urethane, or any other biocompatible material as is commonly used in endoscopes or other medical devices. The sheath coating or material is selected so that it may come in contact with living tissue without causing harm or damage. The electrodes used to control the shape and length of the activated polymer material may, in some embodiments, be insulated or covered to prevent electric shock, which may also be accomplished with biocompatible materials. In other embodiments, the electrodes are compatible electrodes. In one embodiment, the sheath is disposable. In another embodiment, the sheath is cleanable and reusable.

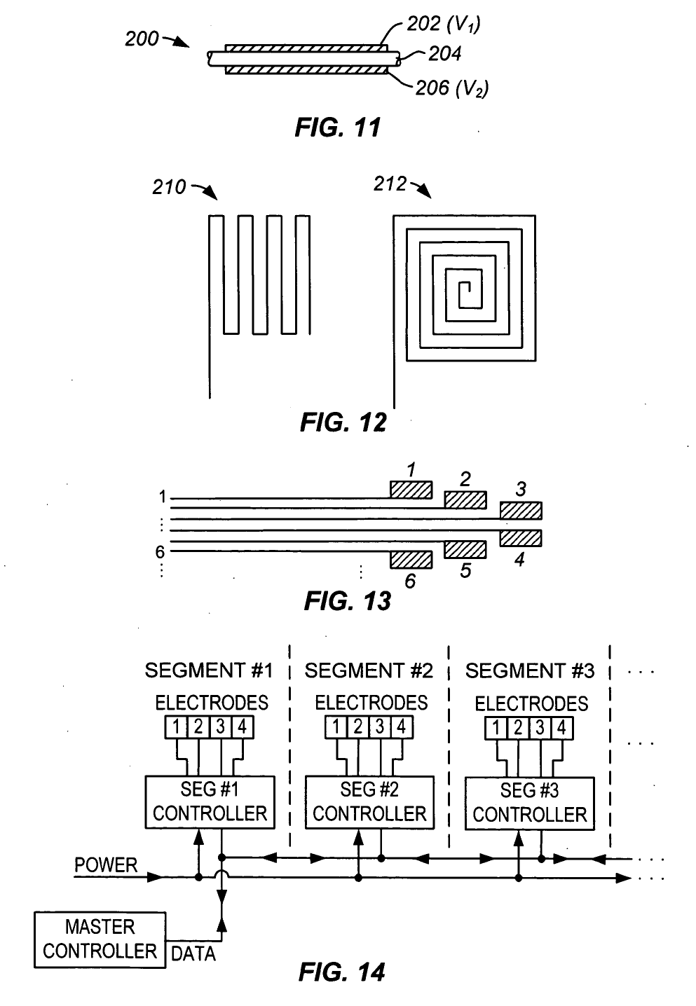

Actuation of the activated polymer material may occur in any of a number of ways depending upon the activation mechanism of that particular polymer. For example, the activation may occur for some polymers by placing them, or parts, or regions of them, in the presence of an electric field. In other cases, an activation mechanism may be related to placing an activated polymer in contact with substances that have varying levels of pH. In some embodiments, electrically activated polymer materials and actuators are actuated through use of electric fields. order to create the electric fields, electrodes may be used, as shown in FIG. 11. These electrodes 202, 206 may be created by placing conductive materials on either side of a piece or region of electro-polymeric material 204, and causing the conductive material 202 on one side of the electro-polymeric material to be at one voltage potential (V₁) while causing the conductive material 206 on the other side of the electro-polymeric material to be at another voltage potential (V₂). In this way, an electric field is established across the electro-polymeric material. The voltage potential may be steady and constant, or may be time-varying.

In another variation, the electrodes may be separate materials in very close contact with the electro-polymeric material. The arrangement of electrodes and electro-polymeric material may be created, e.g., in a sandwich configuration, with each component comprised of a separate piece. The layers may be either flat or tubular. A thin, conductive, flexible material such as Mylar may be used. In order to allow for the contraction, relaxation, and/or expansion of the electro-polymeric material, the layers of the sandwich arrangement may be able to slide relative to each other. For this reason, slippery or lubricious materials may be utilized.

In yet another variation, the electrodes may be bonded directly to the surface of the activated polymer material. In this case, the electrodes are preferably flexible and able to be compressed and expanded so that they may move along with the electro-polymeric material as it is caused to contract, relax and expand. Electrodes made out of flexible material, such as conductive rubber or compliant weaves of conductive material may be used to allow the activated polymer material the maximum range of motion. In some embodiments, flexible methods of attaching the electrodes to the surface of the electro-polymeric material are preferred, such as rubber cement, urethane bonding, or other flexible adhesives. Additional electrode embodiments and compliant electrode embodiments are described in U.S. Pat. No. 6,376,971 to Pelrine et al. entitled, “Electroactive Polymer Electrodes,” the entirety of which is incorporated herein by reference.

In yet another variation, the electrodes may be printed directly onto the surface of an activated polymer material, using a process such as silk-screening with conductive ink, or a reductive process such as is used in the production of printed circuit boards. In this variation, the conductive ink may need to expand and contract along with the movement of the activated polymer material. In order to achieve this, the electrode may be subdivided into regions to allow for gross motions, such as wavy lines or other geometric shapes. FIG. 12 shows patterns 210, 212 of conductive ink that would allow for large degrees of stretching and contracting. In this variation, it may also be desirable to print all connections needed to individually control any or all of the regions of electrodes, so that a large number of regions of activated polymer material may be controlled, thus reducing or eliminating the requirement for additional wiring, as shown in FIG. 13.

Controlling the voltage potential of each of the individually controllable electrodes effects the control of the shape of the pieces or regions of the electro-polymeric material used to control the shape of the articulating instrument. This may be done by use of a controller that switches each of the electrodes on or off, and controls the voltage at each of the electrodes individually to any desired voltage. This may be accomplished by use of a computer or other programmable controller. The controller will then be capable of actuating each individually controllable region, portion, or piece of electro-polymeric material of the endoscope. In this way, the shape of the entire length of the endoscope may be controlled in any way desired, including the “follow-the-leader” algorithm, as described above.

In yet another variation, a separate connection may be made between each of the individual electrodes and a controller. In this variation, a separate wire or pair of wires, or printed trace comprising a wire, may be used to connect each electrode to a controller, such as is shown in the schematic illustration in FIG. 13.

In yet another variation, a network of small controllers that are each capable of switching and controlling a smaller number of electrodes, such as would be required to actuate a single segment of an endoscope, are connected together to a main controller with a data network and a power network, as shown in FIG. 14. The main controller would then configure each of the segments individually by communicating the settings for each of the electrodes to each communications node on the network. This significantly reduces the number of connections that must be made from each electrode to the main controller of the endoscope. Additional controller are described in the incorporated Heim and Pelrine patents and applications as well as US Patent Application publication US 2003/0067245 to Pelrine et al. entitled “Master/Slave Electroactive Polymer Systems,” incorporated herein by reference.

In order to cause the segments, regardless of the variation of design selected, to actuate as quickly and responsively as possible, it may be beneficial to actively pull against regions of electro-polymeric material that have been caused to stop contracting and are in the process of relaxing. This has the benefit of decreasing the response time required for a segment to achieve a newly commanded position, as the time for a region or piece of electro-polymeric material to relax passively is longer than that required for the opposing piece or region of electro-polymeric material to pull the segment to the new required position. Using this algorithm, segments, joints or hinges are actively pulled into new positions, instead of allowing them to relax to achieve new positions.

Before turning to additional alternative structures, fabrication and applications of rolled electroactive polymers as used in some embodiments of the present invention, as well as some of the basic principles of electrically activated or electroactive polymer construction and operation will first be illuminated. The transformation between electrical and mechanical energy in devices of the present invention is based on energy conversion of one or more active areas of an electroactive polymer. Electroactive polymers are capable of converting between mechanical energy and electrical energy. In some cases, an electroactive polymer may change electrical properties (for example, capacitance and resistance) with changing mechanical strain.

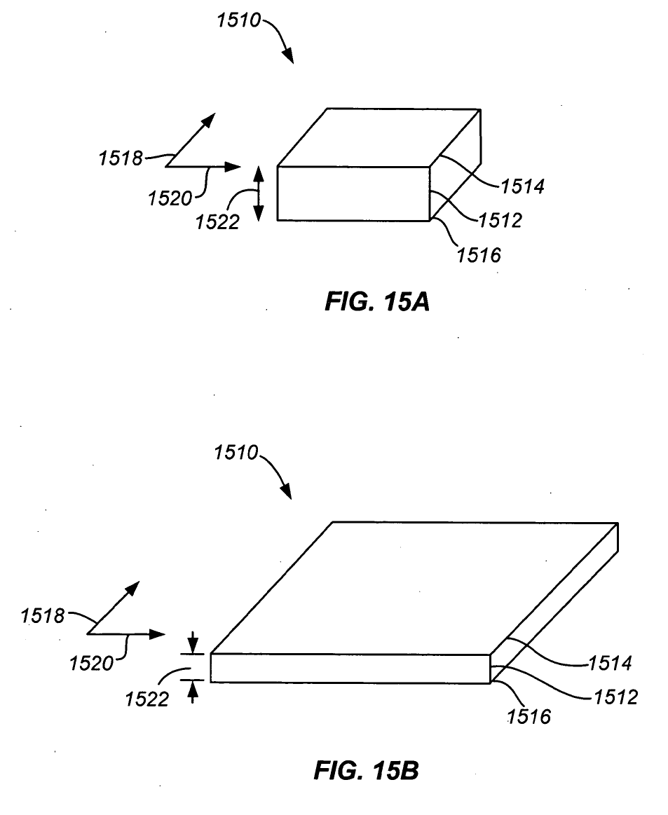

To help illustrate the performance of an electroactive polymer in converting between electrical energy and mechanical energy, FIG. 15A illustrates a top perspective view of a transducer portion 1510 in accordance with one embodiment of the present invention. The transducer portion 1510 comprises a portion of an electroactive polymer 1512 for converting between electrical energy and mechanical energy. In one embodiment, an electroactive polymer refers to a polymer that acts as an insulating dielectric between two electrodes and may deflect upon application of a voltage difference between the two electrodes (a ‘dielectric elastomer’). Top and bottom electrodes 1514 and 1516 are attached to the electroactive polymer 1512 on its top and bottom surfaces, respectively, to provide a voltage difference across polymer 1512, or to receive electrical energy from the polymer 1512. Polymer 1512 may deflect with a change in electric field provided by the top and bottom electrodes 1514 and 1516. Deflection of the transducer portion 1510 in response to a change in electric field provided by the electrodes 1514 and 1516 is referred to as ‘actuation’. Actuation typically involves the conversion of electrical energy to mechanical energy. As polymer 1512 changes in size, the deflection may be used to produce mechanical work.

FIG. 15B illustrates a top perspective view of the transducer portion 1510 including deflection. In general, deflection refers to any displacement, expansion, contraction, torsion, linear or area strain, or any other deformation of a portion of the polymer 1512. For actuation, a change in electric field corresponding to the voltage difference applied to or by the electrodes 1514 and 1516 produces mechanical pressure within polymer 1512. In this case, the unlike electrical charges produced by electrodes 1514 and 1516 attract each other and provide a compressive force between electrodes 1514 and 1516 and an expansion force on polymer 1512 in planar directions 1518 and 1520, causing polymer 1512 to compress between electrodes 1514 and 1516 and stretch in the planar directions 1518 and 1520.

Electrodes 1514 and 1516 are compliant and change shape with polymer 1512. The configuration of polymer 1512 and electrodes 1514 and 1516 provides for increasing polymer 1512 response with deflection. More specifically, as the transducer portion 1510 deflects, compression of polymer 1512 brings the opposite charges of electrodes 1514 and 1516 closer and the stretching of polymer 1512 separates similar charges in each electrode. In one embodiment, one of the electrodes 1514 and 1516 is ground. For actuation, the transducer portion 1510 generally continues to deflect until mechanical forces balance the electrostatic forces driving the deflection. The mechanical forces include elastic restoring forces of the polymer 1512 material, the compliance of electrodes 1514 and 1516, and any external resistance provided by a device and/or load coupled to the transducer portion 1510, etc. The deflection of the transducer portion 1510 as a result of an applied voltage may also depend on a number of other factors such as the polymer 1512 dielectric constant and the size of polymer 1512.

Electroactive polymers in accordance with the present invention are capable of deflection in any direction. After application of a voltage between the electrodes 1514 and 1516, the electroactive polymer 1512 increases in size in both planar directions 1518 and 1520. In some cases, the electroactive polymer 1512 is incompressible, e.g. has a substantially constant volume under stress. In this case, the polymer 1512 decreases in thickness as a result of the expansion in the planar directions 1518 and 1520. It should be noted that the present invention is not limited to incompressible polymers and deflection of the polymer 1512 may not conform to such a simple relationship.

Application of a relatively large voltage difference between electrodes 1514 and 1516 on the transducer portion 1510 shown in FIG. 15A will cause transducer portion 1510 to change to a thinner, larger area shape as shown in FIG. 15B. In this manner, the transducer portion 1510 converts electrical energy to mechanical energy. The transducer portion 1510 may also be used to convert mechanical energy to electrical energy.

For actuation, the transducer portion 1510 generally continues to deflect until mechanical forces balance the electrostatic forces driving the deflection. The mechanical forces include elastic restoring forces of the polymer 1512 material, the compliance of electrodes 1514 and 1516, and any external resistance provided by a device and/or load coupled to the transducer portion 1510, etc. The deflection of the transducer portion 1510 as a result of an applied voltage may also depend on a number of other factors such as the polymer 1512 dielectric constant and the size of polymer 1512.

In one embodiment, electroactive polymer 1512 is pre-strained. Pre-strain of a polymer may be described, in one or more directions, as the change in dimension in a direction after pre-straining relative to the dimension in that direction before pre-straining. The pre-strain may comprise elastic deformation of polymer 1512 and be formed, for example, by stretching the polymer in tension and fixing one or more of the edges while stretched. Alternatively, as will be described in greater detail below, a mechanism such as a spring may be coupled to different portions of an electroactive polymer and provide a force that strains a portion of the polymer. For many polymers, pre-strain improves conversion between electrical and mechanical energy. The improved mechanical response enables greater mechanical work for an electroactive polymer, e.g., larger deflections and actuation pressures. In one embodiment, pre-strain improves the dielectric strength of the polymer. In another embodiment, the pre-strain is elastic. After actuation, an elastically pre-strained polymer could, in principle, be unfixed and return to its original state.

In one embodiment, pre-strain is applied uniformly over a portion of polymer 1512 to produce an isotropic pre-strained polymer. By way of example, an acrylic elastomeric polymer may be stretched by 200 to 400 percent in both planar directions. In another embodiment, pre-strain is applied unequally in different directions for a portion of polymer 1512 to produce an anisotropic pre-strained polymer. In this case, polymer 1512 may deflect greater in one direction than another when actuated. Pre-strain has been earlier described. In one embodiment, the deflection in direction 1518 of transducer portion 1510 can be enhanced by exploiting large pre-strain in the perpendicular direction 1520. For example, an acrylic elastomeric polymer used as the transducer portion 1510 may be stretched by 10 percent in direction 1518 and by 500 percent in the perpendicular direction 1520. The quantity of pre-strain for a polymer may be based on the polymer material and the desired performance of the polymer in an application.

Generally, after the polymer is pre-strained, it may be fixed to one or more objects or mechanisms. For a rigid object, the object is preferably suitably stiff to maintain the level of pre-strain desired in the polymer. A spring or other suitable mechanism that provides a force to strain the polymer may add to any pre-strain previously established in the polymer before attachment to the spring or mechanisms, or may be responsible for all the pre-strain in the polymer. The polymer may be fixed to the one or more objects or mechanisms according to any conventional method known in the art such as a chemical adhesive, an adhesive layer or material, mechanical attachment, etc.

Transducers and pre-strained polymers of the present invention are not limited to any particular rolled geometry or type of deflection. For example, the polymer and electrodes may be formed into any geometry or shape including tubes and multi-layer rolls, rolled polymers attached between multiple rigid structures, rolled polymers attached across a frame of any geometry—including curved or complex geometries, across a frame having one or more joints, etc. Deflection of a transducer according to the present invention includes linear expansion and compression in one or more directions, bending, axial deflection when the polymer is rolled, deflection out of a hole provided on an outer cylindrical around the polymer, etc. Deflection of a transducer may be affected by how the polymer is constrained by a frame or rigid structures attached to the polymer.

Materials suitable for use as an electroactive polymer with the present invention may include any substantially insulating polymer or rubber (or combination thereof) that deforms in response to an electrostatic force or whose deformation results in a change in electric field. One suitable material is NuSil CF19-2186 as provided by NuSil Technology of Carpenteria, Calif. Other exemplary materials suitable for use as a pre-strained polymer include silicone elastomers, acrylic elastomers such as VHB 4910 acrylic elastomer as produced by 3M Corporation of St. Paul, Minn., polyurethanes, thermoplastic elastomers, copolymers comprising PVDF, pressure-sensitive adhesives, fluoroelastomers, polymers comprising silicone and acrylic moieties, and the like. Polymers comprising silicone and acrylic moieties may include copolymers comprising silicone and acrylic moieties, polymer blends comprising a silicone elastomer and an acrylic elastomer, for example. Combinations of some of these materials may also be used as the electroactive polymer as an activated polymer or polymer actuator or transducer of embodiments of articulating instruments of the present invention.

Materials used as an electroactive polymer may be selected based on one or more material properties such as a high electrical breakdown strength, a low modulus of elasticity (for large or small deformations), a high dielectric constant, etc. In one embodiment, the polymer is selected such that is has an elastic modulus at most about 100 MPa. In another embodiment, the polymer is selected such that is has a maximum actuation pressure between about 0.05 MPa and about 10 MPa, and preferably between about 0.3 MPa and about 3 MPa. In another embodiment, the polymer is selected such that is has a dielectric constant between about 2 and about 20, and preferably between about 2.5 and about 12.

An electroactive polymer layer in an actuator of the present invention may have a wide range of thicknesses. In one embodiment, polymer thickness may range between about 1 micrometer and 2 millimeters. Polymer thickness may be reduced by stretching the film in one or both planar directions. In many cases, electroactive polymers of the present invention may be fabricated and implemented as thin films. Thicknesses suitable for these thin films may be below 50 micrometers.

As electroactive polymers of the present invention may deflect at high strains, electrodes attached to the polymers should also deflect without compromising mechanical or electrical performance. Generally, electrodes suitable for use with the present invention may be of any shape and material provided that they are able to supply a suitable voltage to, or receive a suitable voltage from, an electroactive polymer. The voltage may be either constant or varying over time. In one embodiment, the electrodes adhere to a surface of the polymer. Electrodes adhering to the polymer are preferably compliant and conform to the changing shape of the polymer. Correspondingly, the present invention may include compliant electrodes that conform to the shape of an electroactive polymer to which they are attached. The electrodes may be only applied to a portion of an electroactive polymer and define an active area according to their geometry. Several examples of electrodes that only cover a portion of an electroactive polymer will be described in further detail below.

Various types of electrodes suitable for use with the present invention are described in U.S. Pat. No. 6,376,971, which was previously incorporated by reference above. Electrodes described therein and suitable for use with the present invention include structured electrodes comprising metal traces and charge distribution layers, textured electrodes comprising varying out of plane dimensions, conductive greases such as carbon greases or silver greases, colloidal suspensions, high aspect ratio conductive materials such as carbon fibrils and carbon nanotubes, and mixtures of ionically conductive materials. As described herein, embodiments of the articulating instruments of the present invention may advantageously include one or more electrodes, including one or compliant electrodes and one or more active areas for actuating an activated polymer. In one embodiment, the activated polymer in an electrically activated polymer or an electroactive polymer. Generally speaking, electrodes suitable for use with the present invention may be of any shape and material provided they are able to supply or receive a suitable voltage, either constant or varying over time, to or from an activated polymer. In one embodiment, the electrodes adhere to a surface of the polymer. Electrodes adhering to the polymer are preferably compliant and conform to the changing shape of the polymer. In some embodiments, an electrode or a plurality of electrodes may be applied to only a portion of an activated polymer and define an active area according to their geometry. In one specific embodiment, the activated polymer is an electroactive dielectric polymer.

The compliant electrodes are capable of deflection in one or more directions. Linear strain may be used to describe the deflection of a compliant electrode in one of these directions. As the term is used herein, linear strain of a compliant electrode refers to the deflection per unit length along a line of deflection. Maximum linear strains (tensile or compressive) of at least about 50 percent are possible for compliant electrodes of the present invention. For some compliant electrodes, maximum linear strains of at least about 100 percent are common. Of course, an electrode may deflect with a strain less than the maximum. In one embodiment, the compliant electrode is a ‘structured electrode’ that comprises one or more regions of high conductivity and one or more regions of low conductivity.

Materials used for electrodes of the present invention may vary. Suitable materials used in an electrode may include graphite, carbon black, colloidal suspensions, thin metals including silver and gold, silver filled and carbon filled gels and polymers, and ionically or electronically conductive polymers. The compliant electrodes of the present invention may be used alone or in combination with a charge distribution layer. In a specific embodiment, an electrode suitable for use with the present invention comprises 80 percent carbon grease and 20 percent carbon black in a silicone rubber binder such as Stockwell RTV60-CON as produced by Stockwell Rubber Co. Inc. of Philadelphia, Pa. The carbon grease is of the type such as NyoGel 756G as provided by Nye Lubricant Inc. of Fairhaven, Mass. The conductive grease may also be mixed with an elastomer, such as silicon elastomer RTV 118 as produced by General Electric of Waterford, N.Y., to provide a gel-like conductive grease.

In embodiments having a charge distribution layer, the electrodes are considered structured electrodes meaning that pattered conductive traces or portions one either side of an activated polymer are separated from the polymer by a compliant charge distribution layer. As such, the metal traces and charge distribution layer are applied to opposite surfaces of the polymer. Accordingly, a structured electrode refers to an activated polymer actuator having a cross section, from top to bottom, of upper metal or conductive traces, upper charge distribution layer, activated polymer, lower charge distribution layer, lower metal or conductive traces. One of ordinary skill will appreciate that this general structure may be modified as needed to comport with the requirements of a particular activated polymer. For example, if a conductive polymer is used, a suitable electrolyte would be positioned between either or both of the charge distribution layers.

In general, some embodiments of a charge distribution layer have a conductance greater than the electroactive polymer but less than the metal traces. The non-stringent conductivity requirements of the charge distribution layer allow a wide variety of materials to be used. By way of example, the charge distribution layer may comprise carbon black, fluoroelastomer with colloidal silver, a water-based latex rubber emulsion with a small percentage in mass loading of sodium iodide, and polyurethane with tetrathiafulavalene/tetracyanoquinodimethane (TTF/TCNQ) charge transfer complex. These materials are able to form thin uniform layers with even coverage and have a surface conductivity sufficient to conduct the charge between metal traces before substantial charge leaks into the surroundings. In one embodiment, material for the charge distribution layer is selected based on the RC time constant of the activated polymer used in the actuator. By way of example, surface resistivity for the charge distribution layer suitable for some embodiments of the present invention may be in the range of 10⁶-10¹¹ ohms. It should also be noted that in some other embodiments, a charge distribution layer is not used and the metal traces are patterned directly on the polymer. In these embodiments where the charge distribution layer is not used, air or another chemical species on the polymer surface may be sufficient to carry charge between the traces. This effect may be enhanced by increasing the surface conductivity through surface treatments such as plasma etching or ion implantation.

In yet another embodiment, multiple metal electrodes are situated on the same side of a polymer and extend the width of the polymer. In this embodiment, the electrodes provide compliance in the direction perpendicular to width. Two adjacent metal electrodes act as electrodes for polymer material between them. The multiple metal electrodes alternate in this manner and alternating electrodes may be in electrical communication to provide synchronous activation of the polymer. In other embodiments, the electrodes are arranged so as to provide compliance in the direction perpendicular to the length.

It is understood that certain electrode materials may work well with particular polymers and may not work as well for others. By way of example, carbon fibrils work well with acrylic elastomer polymers while not as well with silicone polymers. For most transducers, desirable properties for the compliant electrode may include one or more of the following: low modulus of elasticity, low mechanical damping, low surface resistivity, uniform resistivity, chemical and environmental stability, chemical compatibility with the electroactive polymer, good adherence to the electroactive polymer, and the ability to form smooth surfaces. In some cases, a transducer of the present invention may implement two different types of electrodes, e.g. a different electrode type for each active area or different electrode types on opposing sides of a polymer.

Rolled Electroactive Polymer Devices

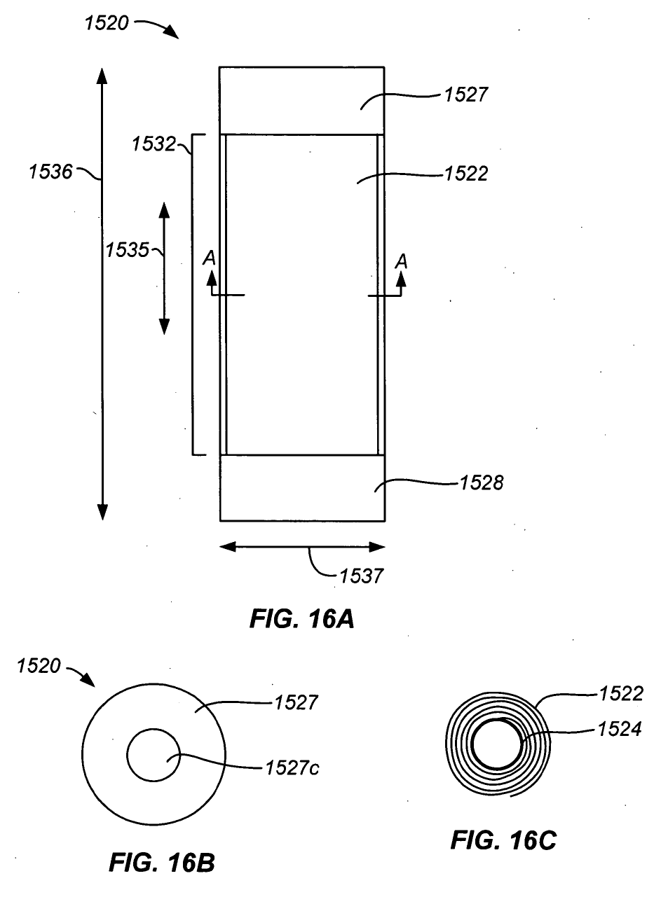

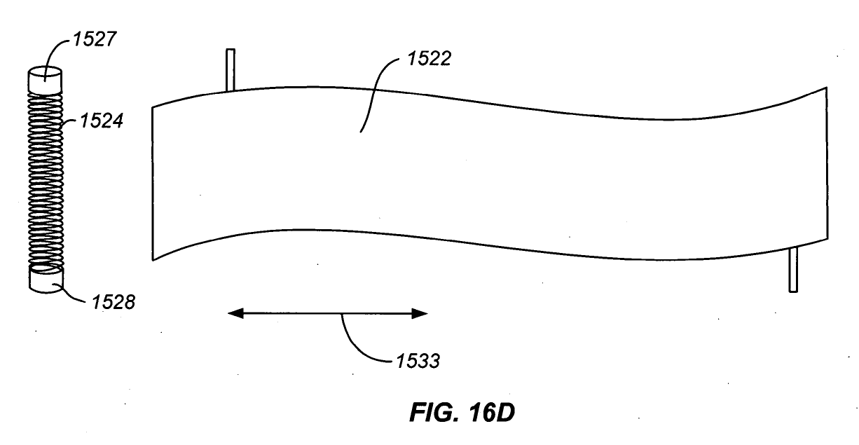

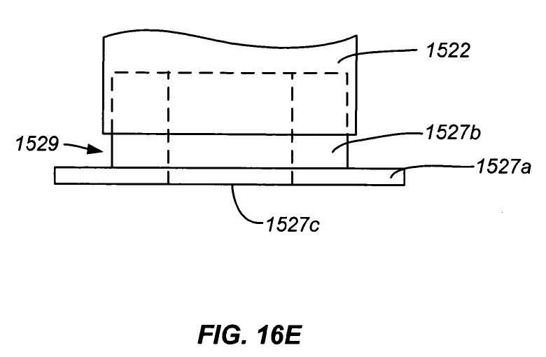

FIGS. 16A-16D show a rolled electroactive polymer device 1520 in accordance with one embodiment of the present invention. FIG. 16A illustrates a side view of device 1520. FIG. 16B illustrates an axial view of device 1520 from the top end. FIG. 16C illustrates an axial view of device 1520 taken through cross section A-A. FIG. 16D illustrates components of device 1520 before rolling. Device 1520 comprises a rolled electroactive polymer 1522, spring 1524, end pieces 1527 and 1528, and various fabrication components used to hold device 1520 together.

As illustrated in FIG. 16C, electroactive polymer 1522 is rolled. In one embodiment, a rolled electroactive polymer refers to an electroactive polymer with, or without electrodes, wrapped round and round onto itself (e.g., like a poster) or wrapped around another object (e.g., spring 1524). The polymer may be wound repeatedly and at the very least comprises an outer layer portion of the polymer overlapping at least an inner layer portion of the polymer. In one embodiment, a rolled electroactive polymer refers to a spirally wound electroactive polymer wrapped around an object or center. As the term is used herein, rolled is independent of how the polymer achieves its rolled configuration.

As illustrated by FIGS. 16C and 16D, electroactive polymer 1522 is rolled around the outside of spring 1524. Spring 1524 provides a force that strains at least a portion of polymer 1522. The top end 1524a of spring 1524 is attached to rigid end piece 1527. Likewise, the bottom end 1524b of spring 1524 is attached to rigid end piece 1528. The top edge 1522a of polymer 1522 (FIG. 16D) is wound about end piece 1527 and attached thereto using a suitable adhesive. The bottom edge 1522b of polymer 1522 is wound about end piece 1528 and attached thereto using an adhesive. Thus, the top end 1524a of spring 1524 is operably coupled to the top edge 1522a of polymer 1522 in that deflection of top end 1524a corresponds to deflection of the top edge 1522a of polymer 1522. Likewise, the bottom end 1524b of spring 1524 is operably coupled to the bottom edge 1522b of polymer 1522 and deflection bottom end 1524b corresponds to deflection of the bottom edge 1522b of polymer 1522. Polymer 1522 and spring 1524 are capable of deflection between their respective bottom top portions.

As mentioned above, many electroactive polymers perform better when pre-strained. For example, some polymers exhibit a higher breakdown electric field strength, electrically actuated strain, and energy density when pre-strained. Spring 1524 of device 1520 provides forces that result in both circumferential and axial pre-strain onto polymer 1522.

Spring 1524 is a compression spring that provides an outward force in opposing axial directions (FIG. 16A) that axially stretches polymer 1522 and strains polymer 1522 in an axial direction. Thus, spring 1524 holds polymer 1522 in tension in axial direction 1535. In one embodiment, polymer 1522 has an axial pre-strain in direction 1535 from about 50 to about 300 percent. As will be described in further detail below for fabrication, device 1520 may be fabricated by rolling a pre-strained electroactive polymer film around spring 1524 while it the spring is compressed. Once released, spring 1524 holds the polymer 1522 in tensile strain to achieve axial pre-strain.

Spring 1524 also maintains circumferential pre-strain on polymer 1522. The pre-strain may be established in polymer 1522 longitudinally in direction 1533 (FIG. 16D) before the polymer is rolled about spring 1524. Techniques to establish pre-strain in this direction during fabrication will be described in greater detail below. Fixing or securing the polymer after rolling, along with the substantially constant outer dimensions for spring 1524, maintains the circumferential pre-strain about spring 1524. In one embodiment, polymer 1522 has a circumferential pre-strain from about 100 to about 500 percent. In many cases, spring 1524 provides forces that result in anisotropic pre-strain on polymer 1522.

End pieces 1527 and 1528 are attached to opposite ends of rolled electroactive polymer 1522 and spring 1524. FIG. 16E illustrates a side view of end piece 1527 in accordance with one embodiment of the present invention. End piece 1527 is a circular structure that comprises an outer flange 1527a, an interface portion 1527b, and an inner hole 1527c. Interface portion 1527b preferably has the same outer diameter as spring 1524. The edges of interface portion 1527b may also be rounded to prevent polymer damage. Inner hole 1527c is circular and passes through the center of end piece 1527, from the top end to the bottom outer end that includes outer flange 27a. In a specific embodiment, end piece 1527 comprises aluminum, magnesium or another machine metal. Inner hole 1527c is defined by a hole machined or similarly fabricated within end piece 1527. In a specific embodiment, end piece 1527 comprises ½ inch end caps with a ⅜ inch inner hole 1527c.

In one embodiment, polymer 1522 does not extend all the way to outer flange 1527a and a gap 1529 is left between the outer portion edge of polymer 1522 and the inside surface of outer flange 1527a. As will be described in further detail below, an adhesive or glue may be added to the rolled electroactive polymer device to maintain its rolled configuration. Gap 1529 provides a dedicated space on end piece 1527 for an adhesive or glue than the buildup to the outer diameter of the rolled device and fix to all polymer layers in the roll to end piece 1527. In a specific embodiment, gap 1529 is between about 0 mm and about 5 mm.

The portions of electroactive polymer 1522 and spring 1524 between end pieces 1527 and 1528 may be considered active to their functional purposes. Thus, end pieces 1527 and 1528 define an active region 1532 of device 1520 (FIG. 16A). End pieces 1527 and 1528 provide a common structure for attachment with spring 1524 and with polymer 1522. In addition, each end piece 1527 and 1528 permits external mechanical and detachable coupling to device 1520. For example, device 1520 may be employed in a robotic application where end piece 1527 is attached to an upstream link in a robot and end piece 1528 is attached to a downstream link in, the robot. Actuation of electroactive polymer 1522 then moves the downstream link relative to the upstream link as determined by the degree of freedom between the two links (e.g., rotation of link 152 about a pin joint on link 1).

In a specific embodiment, inner hole 1527c comprises an internal thread capable of threaded interface with a threaded member, such as a screw or threaded bolt. The internal thread permits detachable mechanical attachment to one end of device 1520. For example, a screw may be threaded into the internal thread within end piece 1527 for external attachment to a robotic element. For detachable mechanical attachment internal to device 1520, a nut or bolt to be threaded into each end piece 1527 and 1528 and pass through the axial core of spring 1524, thereby fixing the two end pieces 1527 and 1528 to each other. This allows device 1520 to be held in any state of deflection, such as a fully compressed state useful during rolling. This may also be useful during storage of device 1520 so that polymer 1522 is not strained in storage.

In one embodiment, a stiff member or linear guide 1530 is disposed within the spring core of spring 1524. Since the polymer 1522 in spring 1524 is substantially compliant between end pieces 1527 and 1528, device 1520 allows for both axial deflection along direction 1535 and bending of polymer 1522 and spring 1524 away from its linear axis (the axis passing through the center of spring 1524). In some embodiments, only axial deflection is desired. Linear guide 1530 prevents bending of device 1520 between end pieces 1527 and 1528 about the linear axis. Preferably, linear guide 1530 does not interfere with the axial deflection of device 1520. For example, linear guide 1530 preferably does not introduce frictional resistance between itself and any portion of spring 1524. With linear guide 1530, or any other suitable constraint that prevents motion outside of axial direction 1535, device 1520 may act as a linear actuator or generator with output strictly in direction 1535. Linear guide 1530 may be comprised of any suitably stiff material such as wood, plastic, metal, etc.

Polymer 1522 is wound repeatedly about spring 1522. For single electroactive polymer layer construction, a rolled electroactive polymer of the present invention may comprise between about 2 and about 200 layers. In this case, a layer refers to the number of polymer films or sheets encountered in a radial cross-section of a rolled polymer. In some cases, a rolled polymer comprises between about 5 and about 100 layers. In a specific embodiment, a rolled electroactive polymer comprises between about 15 and about 50 layers.

In another embodiment, a rolled electroactive polymer employs a multilayer structure. The multilayer structure comprises multiple polymer layers disposed on each other before rolling or winding. For example, a second electroactive polymer layer, without electrodes patterned thereon, may be disposed on an electroactive polymer having electrodes patterned on both sides. The electrode immediately between the two polymers services both polymer surfaces in immediate contact. After rolling, the electrode on the bottom side of the electroded polymer then contacts the top side of the non-electroded polymer. In this manner, the second electroactive polymer with no electrodes patterned thereon uses the two electrodes on the first electroded polymer.

Other multilayer constructions are possible. For example, a multilayer construction may comprise any even number of polymer layers in which the odd number polymer layers are electroded and the even number polymer layers are not. The upper surface of the top non-electroded polymer then relies on the electrode on the bottom of the stack after rolling. Multilayer constructions having 2, 4, 6, 8, etc., are possible this technique. In some cases, the number of layers used in a multilayer construction may be limited by the dimensions of the roll and thickness of polymer layers. As the roll radius decreases, the number of permissible layers typically decrease is well. Regardless of the number of layers used, the rolled transducer is configured such that a given polarity electrode does not touch an electrode of opposite polarity. In one embodiment, multiple layers are each individually electroded and every other polymer layer is flipped before rolling such that electrodes in contact each other after rolling are of a similar voltage or polarity.

The multilayer polymer stack may also comprise more than one type of polymer For example, one or more layers of a second polymer may be used to modify the elasticity or stiffness of the rolled electroactive polymer layers. This polymer may or may not be active in the charging/discharging during the actuation. When a non-active polymer layer is employed, the number of polymer layers may be odd. The second polymer may also be another type of electroactive polymer that varies the performance of the rolled product.

In one embodiment, the outermost layer of a rolled electroactive polymer does not comprise an electrode disposed thereon. This may be done to provide a layer of mechanical protection, or to electrically isolate electrodes on the next inner layer.

Device 1520 provides a compact electroactive polymer device structure and improves overall electroactive polymer device performance over conventional electroactive polymer devices. For example, the multilayer structure of device 1520 modulates the overall spring constant of the device relative to each of the individual polymer layers. In addition, the increased stiffness of the device achieved via spring 1524 increases the stiffness of device 1520 and allows for faster response in actuation, if desired.

In a specific embodiment, spring 1524 is a compression spring such as catalog number 11422 as provided by Century Spring of Los Angeles, Calif. This spring is characterized by a spring force of 0.91 lb/inch and dimensions of 4.38 inch free length, 1.17 inch solid length, 0.360 inch outside diameter, 0.3 inch inside diameter. In this case, rolled electroactive polymer device 1520 has a height 36 from about 5 to about 7 cm, a diameter 1537 of about 0.8 to about 1.2 cm, and an active region between end pieces of about 4 to about 5 cm. The polymer is characterized by a circumferential pre-strain from about 300 to about 500 percent and axial pre-strain (including force contributions by spring 1524) from about 150 to about 250 percent.

Device 1520 has many functional uses. As will be described in further detail below, electroactive polymers of the present invention may be used for actuation of multi-segmented instruments for a variety of medical ands industrial applications as described elsewhere. Thus, device 1520 may also be used in robotic applications for actuation and production of mechanical energy. Alternatively, rolled device 20 may contribute to stiffness and damping control of a robotic link or an articulating segment. Thus, either end piece 1527 or 1528 may be coupled to a potentially moving mechanical link to receive mechanical energy from the link and damp the motion. In this case, polymer 1522 converts this mechanical energy to electrical energy according to techniques described below.

Although device 1520 is illustrated with a single spring 1524 disposed internal to the rolled polymer, it is understood that additional structures such as another spring external to the polymer may also be used to provide strain and pre-strain forces. These external structures may be attached to device 1520 using end pieces 1527 and 1528 for example.

The present invention also encompasses mechanisms, other than a spring, used in a rolled electroactive polymer device to apply a force that strains a rolled polymer. As the term is used herein, a mechanism used to provide strain onto a rolled electroactive polymer generally refers to a system or an arrangement of elements that are capable of providing a force to different portions of a rolled electroactive polymer. In many cases, the mechanism is flexible (e.g., a spring) or has moving parts (e.g., a pneumatic cylinder). The mechanism may also comprises rigid parts (such as a frame for example). Alternatively, compressible materials and foams may be disposed internal to the roll to provide the strain forces and allow for axial deflection.

Generally, the mechanism provides a force that onto the polymer. In one embodiment, the force changes the force vs. deflection characteristics of the device, such as to provide a negative force response, as described below. In another embodiment, the force strains the polymer. This latter case implies that the polymer deflects in response to the force, relative to its deflection state without the effects of the mechanism. This strain may include pre-strain as described above. In one embodiment, the mechanism maintains or adds to any pre-strain previously established in the polymer, such pre-strain provided by a fixture during rolling as described below. In another embodiment, no pre-strain is previously applied in the polymer and the mechanism establishes pre-strain in the polymer.

In one embodiment, the mechanism is another elastomer that is similar or different from the electroactive polymer. For example, this second elastomer may be disposed as a nearly-solid rubber core that is axially compressed before rolling (to provide an axial tensile pre-strain on the electroactive polymer). The elastomer core can have a thin hole for a rigid rod to facilitate the rolling process. If lubricated, the rigid rod may be slid out from the roll after fabrication. One may also make a solid elastomer roll tightly wound with electroactive polymer using a similar technique.

The mechanism and its constituent elements are typically operably coupled to the polymer such that the strain is achieved. This may include fixed or detachable coupling, permanent attachment, etc. In the case of the spring above, operable coupling includes the use of an adhesive, such as glue, that attaches opposite ends of the spring to opposite ends of the polymer. An adhesive is also used to attach the rolled polymer to a frame, if desired. The coupling may be direct or indirect. One of skill in the art is aware of numerous techniques to couple or attach two mechanical structures together, and these techniques are not expansively discussed herein for sake of brevity.

Rolled electroactive polymers of the present invention have numerous advantages. Firstly, these designs provide a multilayer device without having to individually frame each layer; and stack numerous frames. In addition, the cylindrical package provided by these devices is advantageous to some applications where long and cylindrical packaging is advantageous over flat packaging associated with planar electroactive polymer devices. In addition, using a larger number of polymer layers in a roll improves reliability of the device and reduces sensitivity to imperfections and local cracks in any individual polymer layer.

Alternate Rolled Electroactive Polymer Device Designs

Multiple Active Areas

In some cases, electrodes cover a limited portion of an electroactive polymer relative to the total area of the polymer. This may be done to prevent electrical breakdown around the edge of a polymer, to allow for polymer portions to facilitate a rolled construction (e.g., an outside polymer barrier layer), to provide multifunctionality, or to achieve customized deflections for one or more portions of the polymer. As the term is used herein, an active area is defined as a portion of a transducer comprising a portion of an electroactive polymer and one or more electrodes that provide or receive electrical energy to or from the portion. The active area may be used for any of the functions described below. For actuation, the active area includes a portion of polymer having sufficient electrostatic force to enable deflection of the portion. For generation or sensing, the active area includes a portion of polymer having sufficient deflection to enable a change in electrostatic energy. A polymer of the present invention may have multiple active areas.

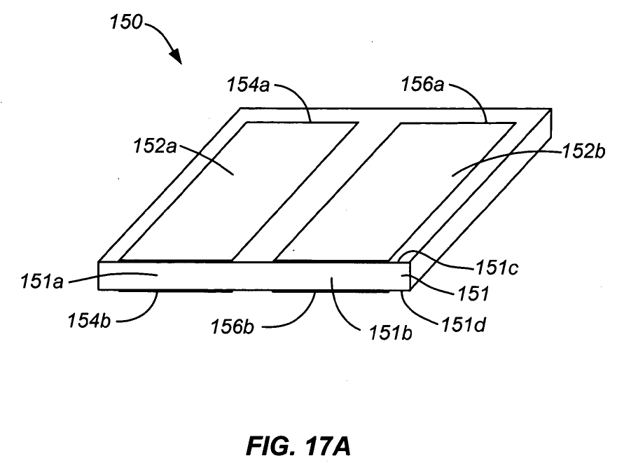

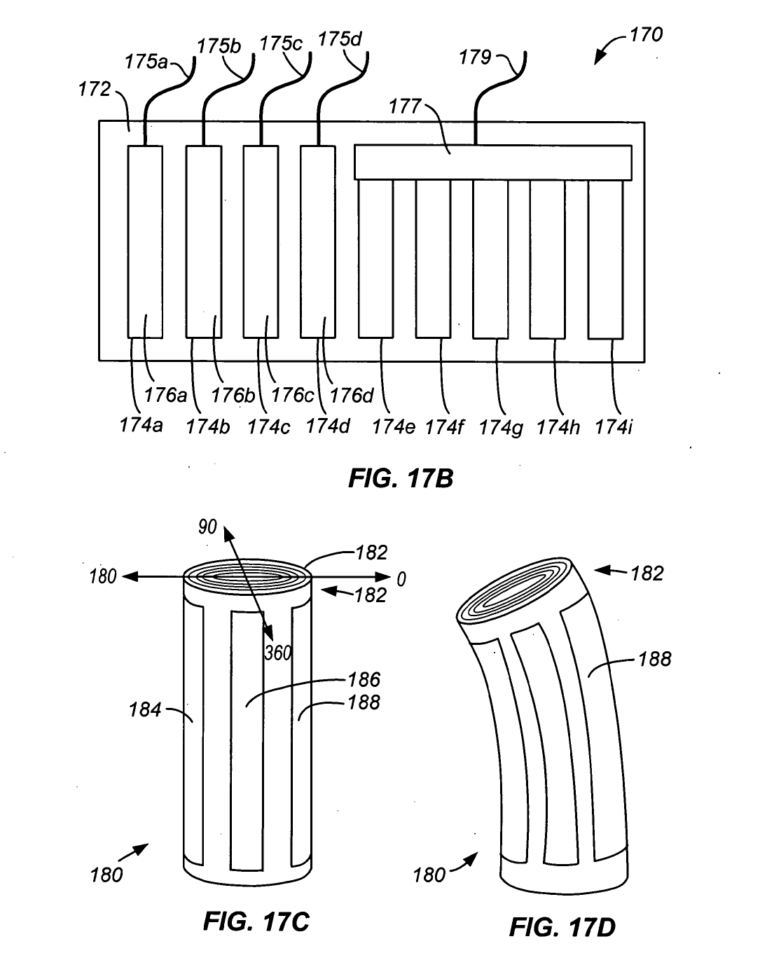

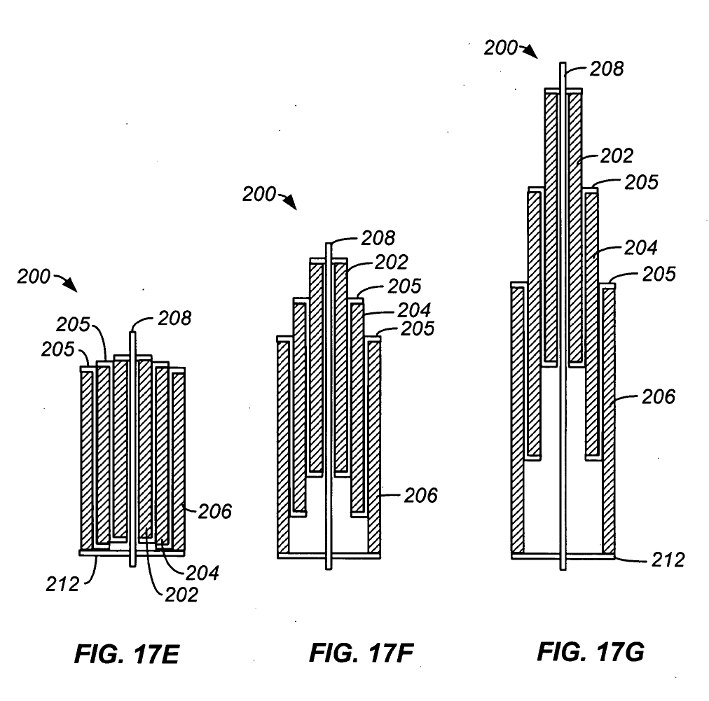

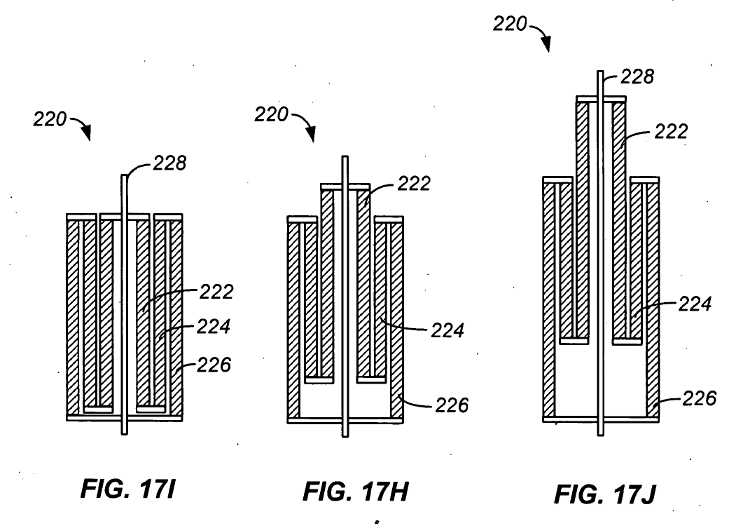

In accordance with the present invention, the term “monolithic” is used herein to refer to electroactive polymers and transducers comprising a plurality of active areas on a single polymer. FIG. 17A illustrates a monolithic transducer 150 comprising a plurality of active areas on a single polymer 151 in accordance with one embodiment of the present invention. The monolithic transducer 150 converts between electrical energy and mechanical energy. The monolithic transducer 150 comprises an electroactive polymer 151 having two active areas 152a and 152b. Polymer 151 may be held in place using, for example, a rigid frame (not shown) attached at the edges of the polymer. Coupled to active areas 152a and 152b are wires 153 that allow electrical communication between active areas 152a and 152b and allow electrical communication with communication electronics 155.

Active area 152a has top and bottom electrodes 154a and 154b that are attached to polymer 151 on its top and bottom surfaces 151c and 151d, respectively. Electrodes 154a and 154b provide or receive electrical energy across a portion 151a of the polymer 151. Portion 151a may deflect with a change in electric field provided by the electrodes 154a and 154b. For actuation, portion 151a comprises the polymer 151 between the electrodes 154a and 154b and any other portions of the polymer 151 having sufficient electrostatic force to enable deflection upon application of voltages using the electrodes 154a and 154b. When active area 152a is used as a generator to convert from electrical energy to mechanical energy, deflection of the portion 151a causes a change in electric field in the portion 151a that is received as a change in voltage difference by the electrodes 154a and 154b.

Active area 152b has top and bottom electrodes 156a and 156b that are attached to the polymer 151 on its top and bottom surfaces 151c and 151d, respectively. Electrodes 156a and 156b provide or receive electrical energy across a portion 151b of the polymer 151. Portion 151b may deflect with a change in electric field provided by the electrodes 156a and 156b. For actuation, portion 151b comprises the polymer 151 between the electrodes 156a and 156b and any other portions of the polymer 151 having sufficient stress induced by the electrostatic force to enable deflection upon application of voltages using the electrodes 156a and 156b. When active area 152b is used as a generator to convert from electrical energy to mechanical energy, deflection of the portion 151b causes a change in electric field in the portion 151b that is received as a change in voltage difference by the electrodes 156a and 156b.

Active areas for an electroactive polymer may be easily patterned and configured using conventional electroactive polymer electrode fabrication techniques. Multiple active area polymers and transducers are further described in Ser. No. 09/779,203, now U.S. Pat. No. 6,664,718 which is incorporated herein by reference for all purposes. Given the ability to pattern and independently control multiple active areas allows rolled transducers of the present invention to be employed in many new applications; as well as employed in existing applications in new ways.

FIG. 17B illustrates a monolithic transducer 170 comprising a plurality of active areas on a single polymer 172, before rolling, in accordance with one embodiment of the present invention. Transducer 170 comprises individual electrodes 174 on the facing polymer side 177. The opposite side of polymer 172 (not shown) may include individual electrodes that correspond in location to electrodes 174, or may include a common electrode that spans in area and services multiple or all electrodes 174 and simplifies electrical communication. Active areas 176 then comprise portions of polymer 172 between each individual electrode 174 and the electrode on the opposite side of polymer 172, as determined by the mode of operation of the active area. For actuation for example, active area 176a for electrode 174a includes a portion of polymer 172 having sufficient electrostatic force to enable deflection of the portion, as described above.

Active areas 176 on transducer 170 may be configured for one or more functions. In one embodiment, all active areas 176 are all configured for actuation. In another embodiment suitable for use with robotic applications, one or two active areas 176 are configured for sensing while the remaining active areas 176 are configured for actuation. In this manner, a rolled electroactive polymer device using transducer 170 is capable of both actuation and sensing. Any active areas designated for sensing may each include dedicated wiring to sensing electronics, as described below.

At shown, electrodes 174a-d each include a wire 175a-d attached thereto that provides dedicated external electrical communication and permits individual control for each active area 176a-d. Electrodes 174e-i are all electrical communication with common electrode 177 and wire 179 that provides common electrical communication with active areas 176e-i. Common electrode 177 simplifies electrical communication with multiple active areas of a rolled electroactive polymer that are employed to operate in a similar manner. In one embodiment, common electrode 177 comprises aluminum foil disposed on polymer 172 before rolling. In one embodiment, common electrode 177 is a patterned electrode of similar material to that used for electrodes 174a-i, e.g., carbon grease.

For example, a set of active areas may be employed for one or more of actuation, generation, sensing, changing the stiffness and/or damping, or a combination thereof. Suitable electrical control also allows a single active area to be used for more than one function. For example, active area 174a may be used for actuation and variable stiffness control of a robotic limb in a robotics application. The same active area may also be used for generation to produce electrical energy based on motion of the robotic limb. Suitable electronics for each of these functions are described in further detail below. Active area 174b may also be flexibly used for actuation, generation, sensing, changing stiffness, or a combination thereof. Energy generated by one active area may be provided to another active area, if desired by an application. Thus, rolled polymers and transducers of the present invention may include active areas used as an actuator to convert from electrical to mechanical energy, a generator to convert from mechanical to electrical energy, a sensor that detects a parameter, or a variable stiffness and/or damping device that is used to control stiffness and/or damping, or combinations thereof.

In one embodiment, multiple active areas employed for actuation are wired in groups to provide graduated electrical control of force and/or deflection output from a rolled electroactive polymer device. For example, a rolled electroactive polymer transducer many have 50 active areas in which 20 active areas are coupled to one common electrode, 10 active areas to a second common electrode, another 10 active areas to a third common electrode, 5 active areas to a fourth common electrode in the remaining five individually wired. Suitable computer management and on-off control for each common electrode then allows graduated force and deflection control for the rolled transducer using only binary on/off switching. The biological analogy of this system is motor units found in many mammalian muscular control systems. Obviously, any number of active areas and common electrodes may be implemented in this manner to provide a suitable mechanical output or graduated control system.

Multiple Degree of Freedom Rolled Devices

In another embodiment, multiple active areas on an electroactive polymer are disposed such subsets of the active areas radially align after rolling. For example, the multiple the active areas may be disposed such that, after rolling, active areas are disposed every 90 degrees in the roll. These radially aligned electrodes may then be actuated in unity to allow multiple degree of freedom motion for a rolled electroactive polymer device.

FIG. 17C illustrates a rolled transducer 180 capable of two-dimensional output in accordance with one environment of the present invention. Transducer 180 comprises an electroactive polymer 182 rolled to provide ten layers. Each layer comprises four radially aligned active areas. The center of each active area is disposed at a 90 degree increment relative to its neighbor. FIG. 17C shows the outermost layer of polymer 182 and radially aligned active areas 184, 186, and 188, which are disposed such that their centers mark 90 degree increments relative to each other. A fourth radially aligned active area (not shown) on the backside of polymer 182 has a center approximately situated 180 degrees from radially aligned active area 186.

Radially aligned active area 184 may include common electrical communication with active areas on inner polymer layers having the same radial alignment. Likewise, the other three radially aligned outer active areas 182, 186, and the back active area not shown, may include common electrical communication with their inner layer counterparts. In one embodiment, transducer 180 comprises four leads that provide common actuation for each of the four radially aligned active area sets.

FIG. 17D illustrates transducer 180 with radially aligned active area 188, and its corresponding radially aligned inner layer active areas, actuated. Actuation of active area 188, and corresponding inner layer active areas, results in axial expansion of transducer 188 on the opposite side of polymer 182. The result is lateral bending of transducer 180, approximately 180 degrees from the center point of active area 188. The effect may also be measured by the deflection of a top portion 189 of transducer 180, which traces a radial arc from the resting position shown in FIG. 17C to his position at shown in FIG. 17D. Varying the amount of electrical energy provided to active area 188, and corresponding inner layer active areas, controls the deflection of the top portion 189 along this arc. Thus, top portion 189 of transducer 180 may have a deflection as shown in FIG. 17D, or greater, or a deflection minimally away from the position shown in FIG. 17C. Similar bending in an another direction may be achieved by actuating any one of the other radially aligned active area sets.

Combining actuation of the radially aligned active area sets produces a two-dimensional space for deflection of top portion 189. For example, radially aligned active area sets 186 and 184 may be actuated simultaneously to produce deflection for the top portion in a 45 degree angle corresponding to the coordinate system shown in FIG. 17C. Decreasing the amount of electrical energy provided to radially aligned active area set 186 and increasing the amount of electrical energy provided to radially aligned active area set 184 moves top portion 189 closer to the zero degree mark. Suitable electrical control then allows top portion 189 to trace a path for any angle from 0 to 360 degrees, or follow variable paths in this two dimensional space.

Transducer 180 is also capable of three-dimensional deflection. Simultaneous actuation of active areas on all four sides of transducer 180 will move top portion 189 upward. In other words, transducer 180 is also a linear actuator capable of axial deflection based on simultaneous actuation of active areas on all sides of transducer 180. Coupling this linear actuation with the differential actuation of radially aligned active areas and their resulting two-dimensional deflection as just described above, results in a three dimensional deflection space for the top portion of transducer 180. Thus, suitable electrical control allows top portion 189 to move both up and down as well as trace two-dimensional paths along this linear axis.

Although transducer 180 is shown for simplicity with four radially aligned active area sets disposed at 90 degree increments, it is understood that transducers of the present invention capable of two- and three-dimensional motion may comprise more complex or alternate designs. For example, eight radially aligned active area sets disposed at 45 degree increments. Alternatively, three radially aligned active area sets disposed at 120 degree increments may be suitable for 2D and 3-D motion.

In addition, although transducer 180 is shown with only one set of axial active areas, the structure of FIG. 17C is modular. In other words, the four radially aligned active area sets disposed at 90 degree increments may occur multiple times in an axial direction. For example, radially aligned active area sets that allow two- and three-dimensional motion may be repeated ten times to provide a snake like robotic manipulator with ten independently controllable links.

Nested Rolled Electroactive Polymer Devices

Some applications desire an increased stroke from a rolled electroactive polymer device. In one embodiment, a nested configuration or a compound rolled activated polymer actuator is used to increase the stroke of an electroactive polymer device. In a nested or compound configuration, one or more electroactive polymer rolls are placed in the hollow central part of another electroactive polymer roll.

FIGS. 17E-G illustrate exemplary cross-sectional views of a nested electroactive polymer device 200, taken through the vertical midpoint of the cylindrical roll, in accordance with one embodiment of the present invention. Nested device 200 comprises three electroactive polymer rolls 202, 204, and 206. Each polymer roll 202, 204, and 206 includes a single active area that provides uniform deflection for each roll. Electrodes for each polymer roll 202, 204, and 206 may be electrically coupled to actuate (or produce electrical energy) in unison, or may be separately wired for independent control and performance. The bottom of electroactive polymer roll 202 is connected to the top of the next outer electroactive polymer roll, namely roll 204, using a connector 205. Connector 205 transfers forces and deflection from one polymer roll to another. Connector 205 preferably does not restrict motion between the rolls and may comprise a low friction and insulating material, such as Teflon. Likewise, the bottom of electroactive polymer roll 204 is connected to the top of the outermost electroactive polymer roll 206. The top of polymer roll 202 is connected to an output shaft 208 that runs through the center of device 200. Although nested device 200 is shown with three concentric electroactive polymer rolls, it is understood that a nested device may comprise another number of electroactive polymer rolls.

Output shaft 208 may provide mechanical output for device 200 (or mechanical interface to external objects). Bearings may be disposed in a bottom housing 212 and allow substantially frictionless linear motion of shaft 208 axially through the center of device 200. Housing 212 is also attached to the bottom of roll 206 and includes bearings that allow travel of shaft 208 through housing 212.

The deflection of shaft 208 comprises a cumulative deflection of each electroactive polymer roll included in nested device 200. More specifically, individual deflections of polymer roll 202, 204 and 206 will sum to provide the total linear motion output of shaft 208. FIG. 17E illustrates nested electroactive polymer device 200 with zero deflection. In this case, each polymer roll 202, 204 and 206 is in an unactuated (rest) position and device 200 is completely contracted. FIG. 17F illustrates nested electroactive polymer device 200 with 20% strain for each polymer roll 202, 204 and 206. Thus, shaft 208 comprises a 60% overall strain relative to the individual length of each roll. Similarly, FIG. 17G illustrates nested electroactive polymer device 200 with 50% strain for each polymer roll 202, 204 and 206. In this case, shaft 208 comprises a 150% overall strain relative to the individual length of each roll. By nesting multiple electroactive polymer rolls inside each other, the strains of individual rolls add up and provide a larger net stroke than would be achieved using a single roll. Nested electroactive polymer rolled devices are then useful for applications requiring large strains and compact packages.

In another embodiment, shaft 208 may be a shaft inside a tube, which allows the roll to expand and contract axially without bending in another direction. While it would be advantageous in some situations to have 208 attached to the top of 202 and running through bearings, shaft 208 could also be two separate pieces: 1) a shaft connected to 212 and protruding axially about ⅘ of the way toward the top of 206, and 2) a tube connected to the top of 206 and protruding axially about ⅘ of the way toward 212, partially enveloping the shaft connected to 212.

FIGS. 17H-J illustrate exemplary vertical cross-sectional views of a nested electroactive polymer device 220 in accordance with another embodiment of the present invention. Nested device 220 comprises three electroactive polymer rolls 222, 224, and 226. Each polymer roll 222, 224, and 226 includes a single active area that provides uniform deflection for each roll.

In this configuration, adjacent electroactive polymer rolls are connected at their common unconnected end. More specifically, the bottom of electroactive polymer roll 222 is connected to the bottom of the next outer electroactive polymer roll, namely roll 224. Likewise, the top of electroactive polymer roll 224 is connected to the top of the outermost electroactive polymer roll 226. The top of polymer roll 222 is connected to an output shaft 228 that runs through the center of device 220. Similar to as that described with respect to shaft 208, shaft 222 may be a shaft inside a tube, which allows the roll to expand and contract axially without bending in another direction.

FIG. 17H shows the unactuated (rest) position of device 220. FIG. 17I shows a contracted position of device 220 via actuation of polymer roll 224. FIG. 17J shows an extended position of device 220 via actuation of polymer rolls 222 and 226. In the unactuated (rest) position of FIG. 1711, the shaft 208 position will be somewhere between the contracted position of FIG. 17I and the extended position of FIG. 17J, depending on the axial lengths of each individual roll.

This nested design may be repeated with an increasing number of layers to provide increased deflection. Actuating every other roll—starting from the first nested roll—causes shaft 228 to contract. Actuating every other roll—starting from the outermost roll—causes shaft 228 to extend. One benefit to the design of nested device 220 is that charge may be shunted from one polymer roll to another, thus conserving overall energy usage.

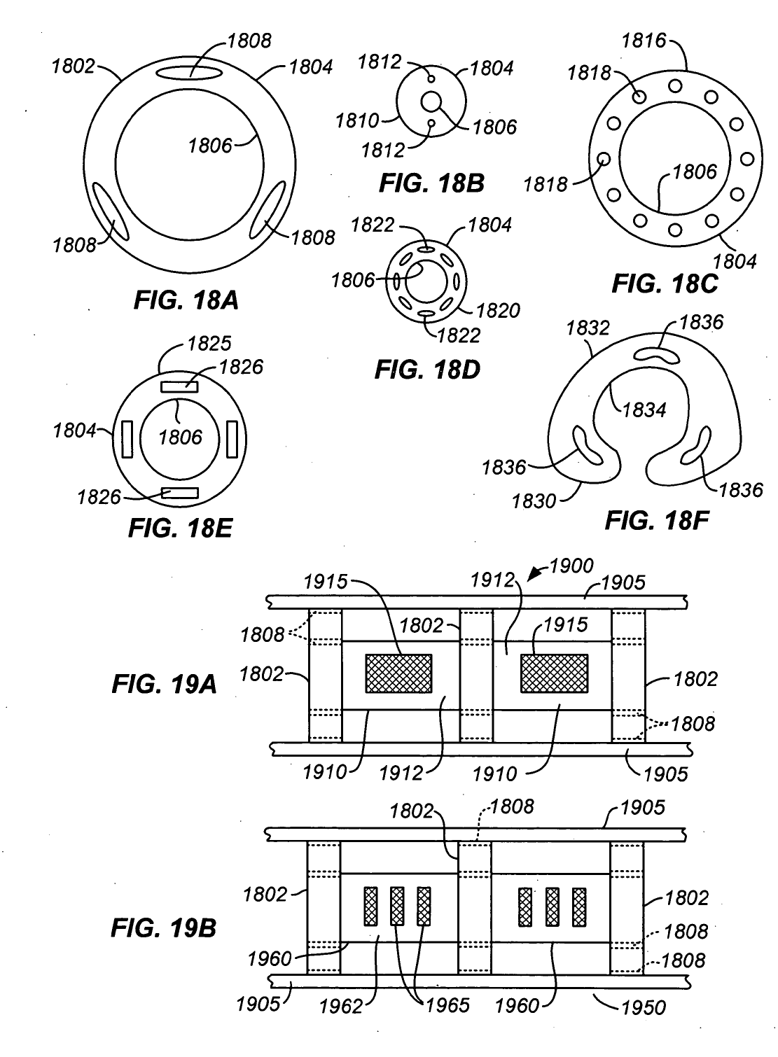

A number of alternative segment embodiments will now be described with regard to FIGS. 18A-18F. In some embodiments there is provided an articulating instrument having at least two segments, each segment having an outer surface and an inner surface and comprising at least two internal actuator access ports disposed between the outer surface and the inner surface. In addition, at least one electromechanical actuator extending through each of the internal actuator access ports and coupled to the at least two segments so that actuation of the at least one electromechanical actuator results in deflection between the at least two segments.

Segment 1802 is an example of an annular and continuous segment having an outer surface 1804 and an inner surface 1806 (FIG. 18A). Three internal actuator access ports 1808 are disposed between the outer surface 1804 and the inner surface 1806. The internal access ports 1808 have, in this embodiment, a generally oval or elliptical shape. Other shapes are possible. As will be described in greater detail below, embodiments of the internal access ports provide an attachment point between the segment and an activated polymer component such as an actuator, a rolled actuator, a sheet of activated polymer material having one or more active areas.

Segment 1810 is generally circular in shape and has an outer surface 1804 and an inner surface 1806 (FIG. 18B). Two internal actuator access ports 1812 are disposed between the outer surface 1804 and the inner surface 1806. The internal access ports 1812 have, in this embodiment, a generally circular shape.

Segment 1816 is generally circular in shape and has an outer surface 1804 and an inner surface 1806 (FIG. 18C). Twelve evenly spaced actuator access ports 1818 are disposed between the outer surface 1804 and the inner surface 1806 and about the circumference of the segment 1816. The internal access ports 1818 have, in this embodiment, a generally circular shape. The shape of each internal access port need not be the same for every port in a given segment and the ports need not be evenly arrayed about the segment. Some ports may be closer to the outer surface 1804 or the inner surface 1806 or two or more ports could be positioned along the same radius and distributed between the inner surface 1806 and the outer surface 1816. While these alternatives are described in relation to an embodiment of segment 1816, they apply as well to the other segment embodiments described herein.

Segment 1820 is generally circular in shape and has an outer surface 1804 and an inner surface 1806 (FIG. 18D). Eight actuator access ports 1822 are arrayed about the segment perimeter between the outer surface 1804 and the inner surface 1806. The internal access ports 1818 have, in this embodiment, a variety of generally oval shapes.

Segment 1825 is generally circular in shape and has an outer surface 1804 and an inner surface 1806 (FIG. 18E). Four actuator access ports 1826 are disposed between the outer surface 1804 and the inner surface 1806 about the circumference of the segment 1825. The internal access ports 1826 have, in this embodiment, a rectangular shape.

Segment 1830 is generally circular and, unlike the earlier segment embodiments, is non-continuous (FIG. 18F). Segment 1830 has an outer surface 1832 and an inner surface 1834. Three actuator access ports 1836 are disposed between the outer surface 1832 and the inner surface 1834 and about the segment 1830. The internal access ports 1836 have, in this embodiment, a compound geometric shape. In this embodiment, the compound geometric shape resembles the shape of a kidney bean. As described below, compound geometric shaped access ports may provide advantageous curvatures for sheets or sections or segments of activated polymer material. Segment 1832 also illustrates a non-annular or non-circular segment shape. Portions of the segment are flared to provide a more oval shape in some embodiments and in other embodiments the shape may resemble a flattened triangle or rounded conical shape.

It is to be appreciated from the above discussion of the various segments and access ports that at least one of the access ports in a segment has a regular geometric shape. In some embodiments, an access ports has a regular geometric shape selected from the group consisting of: circle, rectangle, oval, ellipse. In other embodiments, an access port may have a compound geometric shape. Additionally, the internal access ports could be of any shape, number, orientation and spatial arrangement with without uniform spacing. For example, in an embodiment where an embodiment of a segment is advantageously combined with a pre-bias shape instrument described above, the segment access ports may be distributed in a manner than recognizes the need for actuators to be positioned to counteract the pre-bias shape. In other embodiments, more than one activated polymer actuator or material is provided through, coupled to or terminated in an access port.

FIGS. 19A and 19B illustrate additional embodiment of activated polymer segments that may be used to articulate, bend or otherwise manipulate embodiments of the articulated instruments of the present invention. Articulating segment 1900 and 1950 share a similar construction. These are least two segments, each segment having an outer surface and an inner surface and comprising at least two internal actuator access ports disposed between the outer surface and the inner surface. The illustrated embodiments show segment 1802 with access ports 1808. it is to be appreciated that any of the other described segments or the like may also be used. The articulating segments also include at least one electromechanical actuator extending through each of the internal actuator access ports and coupled to the at least two segments so that actuation of the at least one electromechanical actuator results in deflection between the at least two segments. In one embodiment, the activated polymer actuator 1910 is attached to (i.e. terminates) the outer segments 1802 and passes through and is coupled sufficiently to the middle segment 1802 to allow deflection between each, any and/or all of the segments 1802. In the embodiment illustrated in FIG. 19A, the activated polymer actuator 1910 includes a polymer sheet 1910 and an active area 1915 including an electrode. The polymer sheet may be formed from an activated polymer that has only a portion used in the active area 1915. It is to be appreciated that rather than requiring an additional backing sheet of a different material, the activated polymer material could be used as the structural sheet 1912 used for the actuator.

In addition, a sheath 1905 is attached to the outer surface 1816 of the at least two segments. In an alternative embodiment, the sheath 1905 is attached to the inner surface 1806 of the at least two segments. In some embodiments, the sheath is formed from a suitable material known in the medical arts that is durable, flexible and washable so that it may be reused. In other embodiments, the sheath is removable from the segments and disposable. In yet another embodiment, the sheath material comprises a biocompatible material.

Articulating segment 1950 (FIG. 19B) differs from articulating segment 1900 in that multiple active areas 1965 are provided between segments 1802. Three active areas 1965 are shown in FIG. 19B. More are possible. Moreover, the active areas need not be evenly spaced nor aligned only along the longitudinal axis of the segments. In addition, for all embodiments of segments 1900, 1950, the structure of the active areas and the polymer sheets 1912, 1962 may include pre-strained and unstrained polymers, multi-laminated electrode structures, compliant electrodes, other structural elements to provide for the proper operation of an activated polymer actuator. For example, providing an electrolyte adjacent a conductive polymer type actuator.

While the segments depicted above are closed loops and open loops, the segments may also be used in combination with or replaced by tubes of various lengths if desired. For example, a series of short tubes constructed in a fashion similar to known vascular, biliary or esophageal stents can be used. Such a structure may include the placement of a plurality of actuators positioned between a series of short stent-like elements.

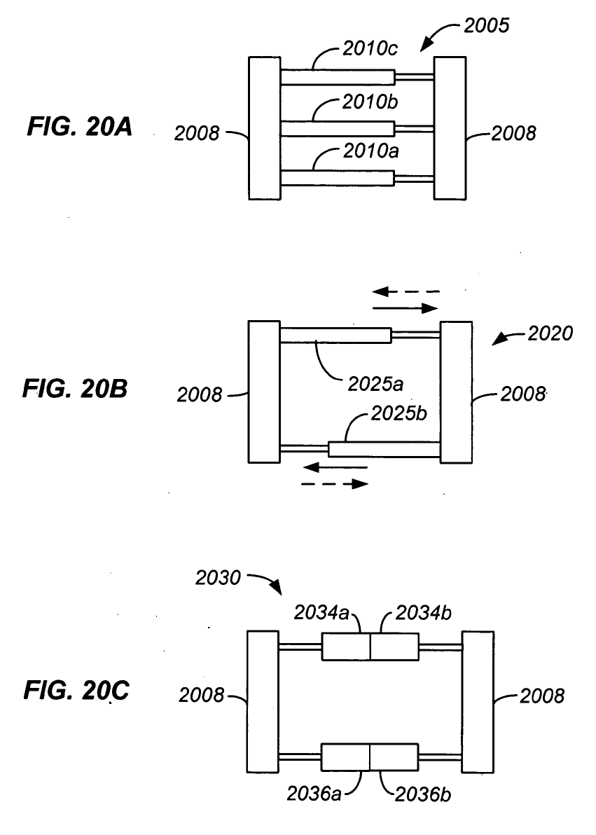

In some embodiments of the present invention, the articulating instrument is actuated, bend or otherwise manipulated using embodiments of the rolled polymer actuators described above. In general, the rolled polymer actuators are extended between a pair of segments 2008. In FIG. 20A, activated segment 2005 includes rolled polymer actuators 2010a, b, and c distributed between the segments 2008. Suitable electronic controls are provided allowing the actuators to be operated separately or in combination to produce the desired deflections between the segments 2008.

Activated segment 2020 includes a cooperative pair of rolled polymers actuators 2025a. and 2025b (FIG. 20B). Rolled, actuators 2025a, 2025b also illustrate how the potential applied to the actuator may be reversed to provide reversible operation. For example, the solid lines indicate application of positive potential and the dashed lines represent the application of negative potential. Suitable electronic controls are provided allowing the actuators to be operated using reversible actuation separately or in combination to produce the desired deflections between the segments 2008.

Activated segment 2030 includes an alternative embodiment of a cooperative rolled polymer actuator pair. Rolled actuator pairs 2034a,b and 2036 a, b are disposed between segments 2008. In one embodiment, the segments 2008 may be manipulated or articulated by having the actuator 2034b push on its attached segment 2008 while the actuator 2034a pulls on its attached segment 2008. In another embodiment, both actuator pairs 2034 a,b and 2036 a,b are operating in the above described push-pull mode. In another embodiment, less than all the actuators are activated to deflect the segments 2008. Other alternative rolled activated polymer actuator configurations are possible. For example, the reversible aspect described in FIG. 20B may be applied to other embodiments, and combinations of actuator configurations 2010, 2025 and 2034 may be used between the same segment pair.

Further to the embodiments described in FIGS. 5, 6, 7, 8 and 9, a single elongated tube 2100 can be used as a structural element to form an embodiment of an articulating instrument of the present invention. In some embodiments, the design of the structure may also be in the form of a plurality of stent-like elements. In some embodiments, the elongate member 2100 is formed from a flexible or elastic material such that the member 2100 can be configured so that it will possess an inherent bias or memory such as discussed above in FIGS. 2e and 2f. The bias acts to restore the assembly to a substantially linear configuration as illustrated or into any desired bias shape as discussed above. Similarly, actuators coupled to the member 2100 can then be used to deflect it from an original or bias configuration as needed to reflect, for example, the shape of a lumen, organ or body cavity into which the articulating instrument is inserted. Of course, a source of bias such as an elastic sleeve (i.e., inserted within or about the structure as discussed above) may also be provided.

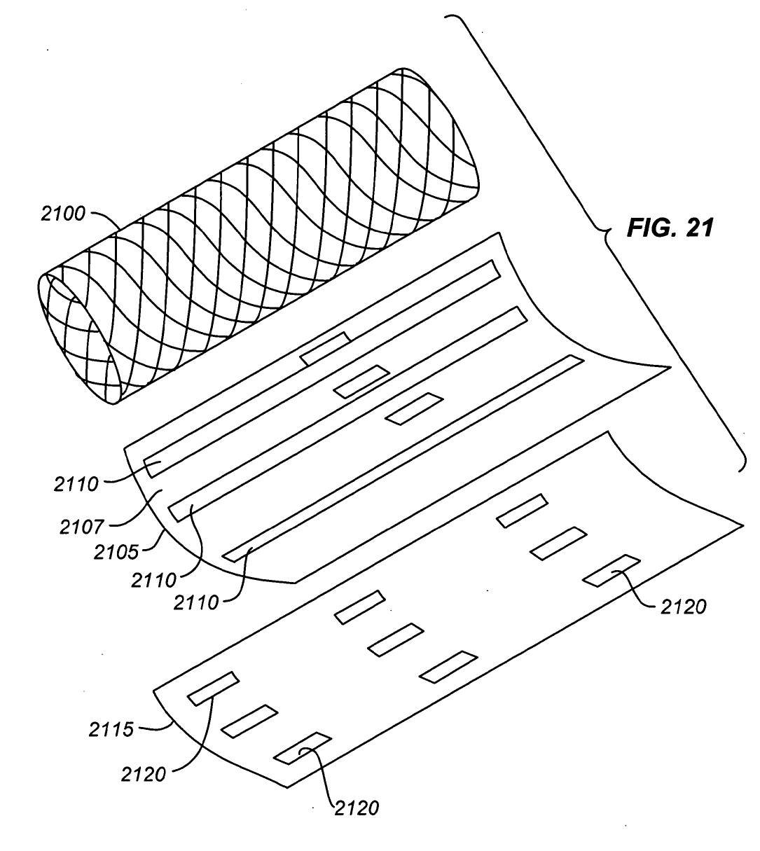

FIG. 21 also illustrates a number of active polymer sheet 2105 having active areas 2110 disposed along a polymer layer 2107. In this embodiment, the polymer sheet 2107 is sufficiently wide to wrap around the member 2100 at least once and, in some embodiments, multiple times. In alternative embodiments discussed elsewhere, the polymer sheet may have multiple active areas but only be as wide as section or portion of the perimeter of the member 2100. In these alternatives, one or more of the polymer sheet sections are utilized to bend or otherwise manipulate the member 2100.

In the illustrated embodiment the active areas extend along the longitudinal axis of the polymer layer 2107. The polymer layer 2107 may advantageously be formed from an activated polymer wherein the active regions are integral to the polymer sheet. The active areas could be in any arrangement, location or orientation as desired since the entire polymer sheet may be used for actuation. This is one advantage other polymer actuators designs that use non-activated polymers or simply a polymer structural element without regard for the inherent simplicity of this design. It is to be appreciated that the active areas 2110 need not be a single monolithic structure but may include serpentine, zigzag or other patterned conductive traces. It is also to be appreciated that embodiments of the active areas 2110 include all of the various alternative electrode and active area configurations described above.

Also illustrated in FIG. 21 are a plurality of strain gauges or feedback polymer elements 2120 provided on a second polymer sheet 2115. The feedback elements may be used to monitor and provide feedback during the manipulation of a segment. In some embodiments, the feedback elements are printed on the sheet 2115. In other embodiments, the feedback elements are electroactive polymer sensors as further described in U.S. Patent Application Publication US 2002/0130673 to Pelrine et al., the entirety of which is incorporated herein by reference. It is to be appreciated that the order of the polymer sheets 2107, 2115 may be altered from the illustrated embodiment where sheet 2107 contacts the member 2100 and sheet 2115 contacts the outside of the sheet 2107. In one alternative embodiment, the sheet 2115 is against between the member 2100 and the sheet 2107. In an alternative embodiment, the sheets 2207, 2115 could be disposed inside member 2100, in any arrangement.

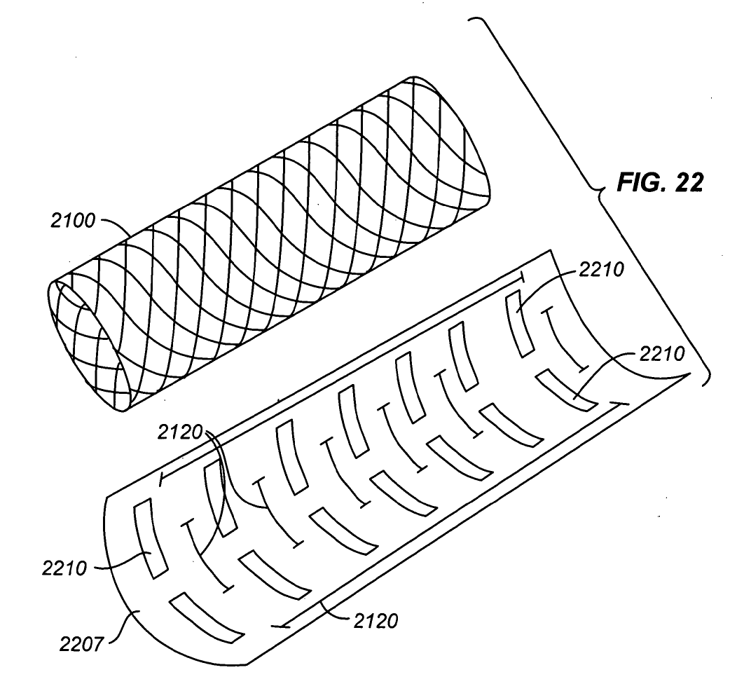

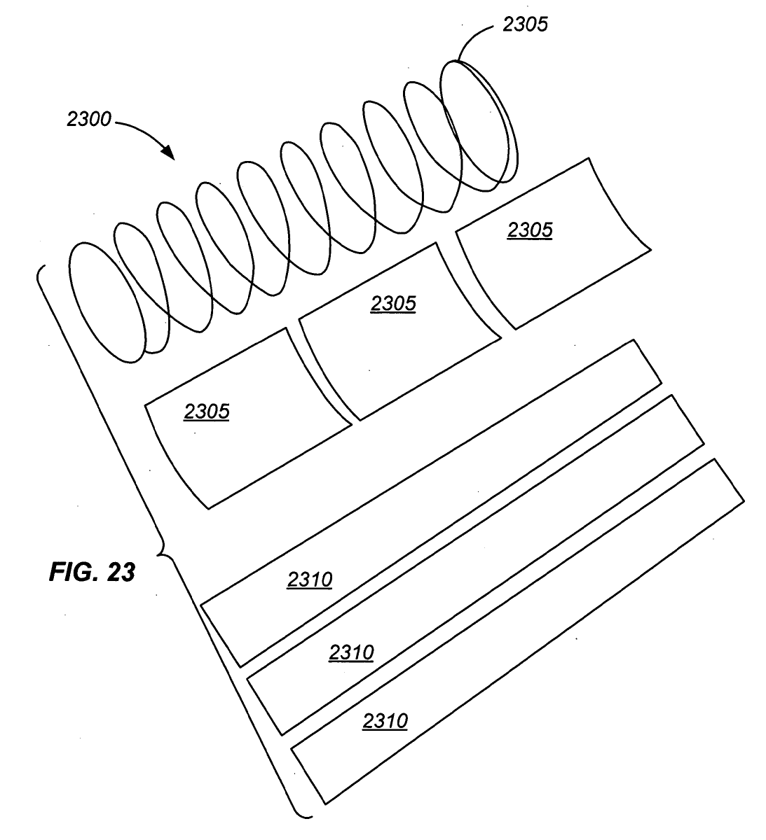

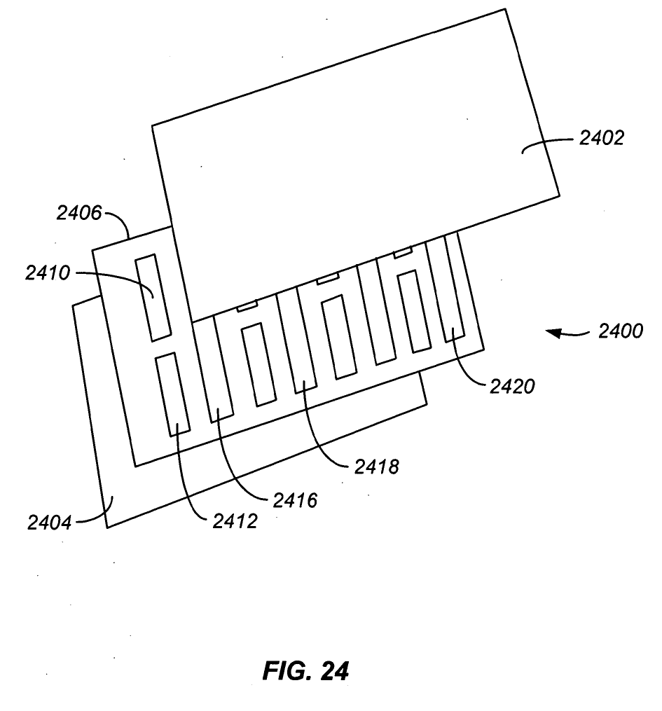

FIG. 22 illustrates anther embodiment of an actuated member 2100. This embodiment differs from the embodiment of FIG. 21 in that a single polymer sheet 2207 is used that included both the active areas 2210 and strain gauges 2120. In addition, the active areas 2210 are aligned nearly orthogonal to the longitudinal axis of the member 2100 in contrast to the longitudinal active areas in FIG. 21. In an alternative embodiment, the sheet 2207 could be disposed inside member 2100.

FIG. 23 illustrates an embodiment of an active polymer actuated segment 2300 according to the present invention. In this embodiment, a coil, or coil tube 2305 defines the segment. Here, compound actuator segments are formed in a laminated structure. A first set of actuators 2305 having an active area (not shown) are provided in a series of hoop structures acting circumferentially, in one embodiment, about the coil 2300. A second set of actuators 2310 are provided that act, in one embodiment, longitudinally on the coil 2300. Each of the actuators 2305, 2310 may include multiple active areas resulting a highly configurable and bendable instrument. Each of the active areas may include all or some of the electrode and/or active area features described above. For example, articulation of the segment 2305 may result from the combination of actuation force(s) generated from one or more active areas in the first set of actuators 2305 with actuation force(s) generated from one or more active areas in the first set of actuators 2310. In an alternative embodiment, the first set of actuators 2305 are provided on a single polymer sheet and the second set of actuators 2310 are provided on a second polymer sheet bonded or coupled to the sheet containing the actuators 2305.

The concept of compound laminate polymer actuators is further illustrated through reference to FIG. 24. Compound laminate polymer actuators 2400 includes polymer layers 2402, 2404 about an activated polymer sheet 2406 having multiple, different active areas 2410, 2412, 2416, 2418, and 2420. In one embodiment, layers 2402, 2404 and 2406 are all activated polymers the only difference is that layer 2406 has multiple active areas. Each of the active areas may include all or some of the electrode and/or active area features described above.

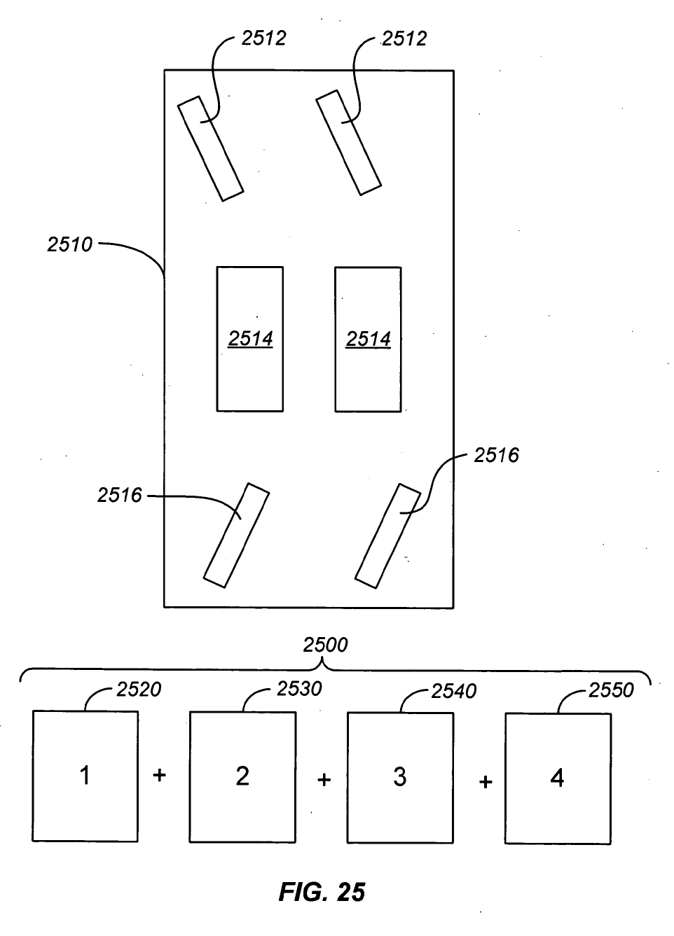

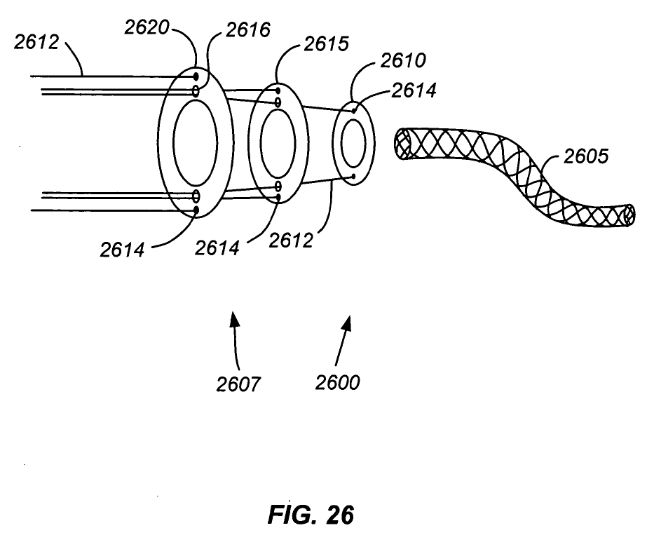

The concept of compound laminate polymer actuators is further illustrated through reference to FIG. 25. In one embodiment, the compound laminate polymer actuator 2500 includes four active polymer layers 2520, 2530, 2540 and 2550 each having multiple, different active areas. In still further embodiments, the orientation of the active areas of each layer may, be different. For example, the active areas in sheet 2520 provide configuration 1, sheet 2530 provides configuration 2 and so forth. Illustrative active polymer sheet 2510 illustrates the point where multiple active areas with different orientations are provided. Active areas 2514 in a generally longitudinal aspect with active areas 2512, 2516 illustrating an active area having complementary angular orientations. Other active area orientations are possible. For example, each of the active area configurations 1 through 4 may be the same, different, or complementary. In one embodiment, the active areas in one sheet operate in a complementary fashion with the active areas in another sheet. In an alternative embodiment, the sheets are adjacent one another. In yet another alternative embodiment, at least one other sheet separates the complementary sheets. While described as sheets it is to be appreciated that the compound laminate polymer actuators of the present invention may be formed into hoops, rings, longitudinal sections, or other partial segments.

Additional active area configurations are possible. For example, an active area may be provided on an activated polymer sheet that produces one or both planar directions of active polymer deformation. Advantageously, multiple active areas and their respective electrodes (with or without conductive layers) may be patterned onto a single active polymer substrate or sheet r material to produce multiple degrees of freedom or actuation modalities from a single activated polymer substrate or sheet.

In some embodiments of the present invention, the articulating instrument is manipulated, bent or controlled using hybrid actuation mechanisms. Hybrid articulating instrument 2600 includes tendon driven segment portion 2607 and an activated polymer portion 2605. For clarity, a sheath or other structural connections that join the two portions have been omitted. The tendon driven segment 2607 includes a plurality of segments here three (2610, 2615, and 2620). Each of the segments includes an attachment point 2614 and all but the distal most segment 2610 include pass thru or portals 2616 allowing force transmission elements 2612 (i.e., tendons, Bowden cables and the like) to attach to more distal segments. Additional details regarding the driven section 2607 may be found in commonly owned and assigned patent application Ser. No. 10/229,577 entitled “Tendon Driven Endoscope and Methods of Insertion,” the entirety of which is incorporated herein by reference. The activated polymer portion 2605 may include any one the activated polymer actuators or configurations described herein. In one embodiment, the segmented articulating instrument includes a selectively steerable distal end actuated by an activated polymer and an automatically controllable proximal end actuated through the use of the force transmission elements, cables and the like. Further still, a curve in a pathway is selected and defined by the shape of selectively steerable distal end actuated by an activated polymer and then automatically propagated along the automatically controllable proximal end actuated through the use of the force transmission elements. It is to be appreciated that the hybrid embodiment includes suitable control systems to provide “follow the leader” type actuation of the hybrid articulating instrument 2600. Additional details of the follow the leader scheme are described in the earlier incorporated Belson U.S. Pat. Nos. 6,468,203 and 6,610,007.

Specific mention has been made to the articulating instrument being a segmented endoscope and other assemblies have been described for use with colonoscopes. It is to be appreciated that the types and specific designs of electromechanical actuators and electromechanical actuator assemblies of embodiments of the present invention may be configured for manipulating a wide variety of controllable articles in the a number of other medical and industrial applications. In addition, embodiments of the present invention can also be configured for use with wireless endoscopes, robotic endoscopes, catheters, specific designed for use catheters such as, for example, thrombolysis catheters, electrophysiology catheters and guide catheters, cannulas, surgical instruments or introducer sheaths or procedure specific articulating instruments such as those used in a variety of medical procedures that use the principals of the embodiments of the invention for navigating within the body, selectively with the body cavity around or between body organs, within body organs and/or through body channels.

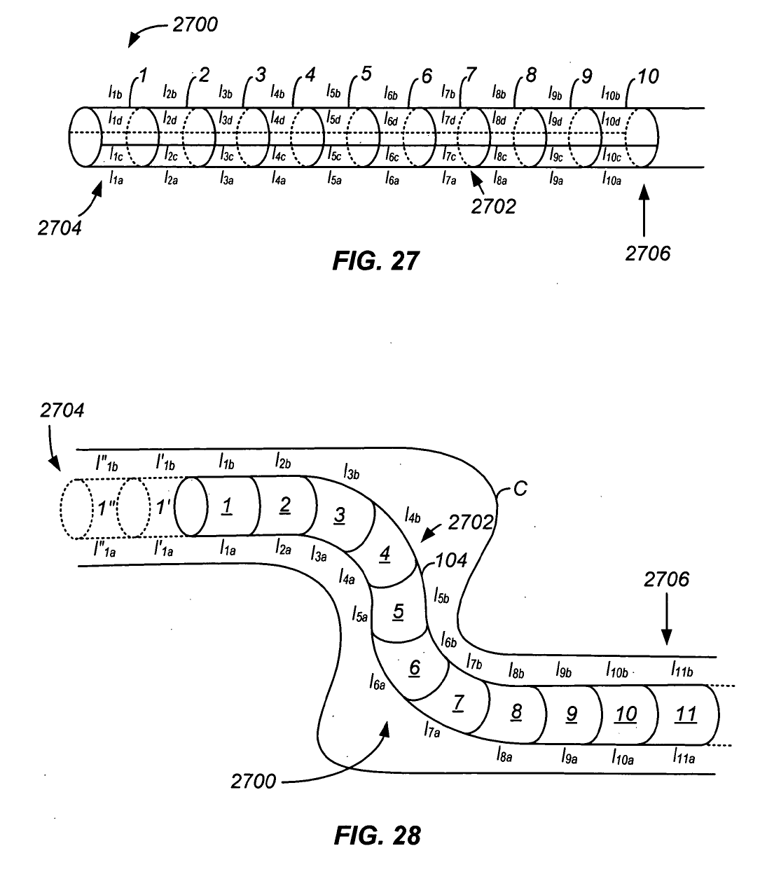

An example of “follow the leader” type control will now be described through reference to FIGS. 27 and 28. Additional details of “follow the leader” type control may be found in U.S. Pat. No. 6,468,203 to Belson (previously incorporated herein by reference).

FIG. 27 shows a wire frame model of a section of the body 2702 of an articulating instrument 2700. While embodiments of the pre-bias shape described herein, this example will address the use of follow the leader in a section, as illustrated, having a straight or unbiased position. Most of the internal structure of the articulating instrument body 2702 has been eliminated in this drawing for the sake of clarity. The articulating instrument body 2702 is divided up into segments or sections 1, 2, 3 . . . 10, etc. The geometry of each section is defined by a suitable number of length measurements or other indications of the relative positions of the various segments. The geometry of a section may be defined using length measurements or other indications. In this illustrative example, the segments will be described as having measurement or indications along 4 axes, namely, the a, b, c and d axes. Fewer axes such as 2 or three as well as more axes may also be used to describe the segments. In this illustrative example, the geometry of section 1 is defined by the four length measurements 1.sub.1a, 1.sub.1b, 1.sub.1c, 1.sub.1d, and the geometry of section 2 is defined by the four length measurements 1.sub.2a, 1.sub.2b, 1.sub.2c, 1.sub.2d, etc. Preferably, each of the length measurements or other indication of segment geometry is individually controlled by a linear actuator, such as through the use of active polymer actuators and materials described herein. The linear actuators may utilize one of several different operating principles. For example, each of the linear actuators may be a self-heating NiTi alloy linear actuator or an electrorheological plastic actuator, or other known mechanical, pneumatic, hydraulic or electromechanical actuator. In some embodiments, other known electromechanical actuators include the active polymer actuators embodiments described herein. Remaining with the illustrative example, the geometry of each section may be altered using the linear actuators to change the four length measurements along the a, b, c and d axes. In some embodiments, the length measurements or other indication of segment geometry are changed in complementary pairs to selectively bend the articulating instrument body 2702 in a desired direction. For example, to bend the articulating instrument body 2702 in the direction of the a axis, the measurements 1.sub.1a, 1.sub.2a, 1.sub.3a . . . 1.sub.10a would be shortened and the measurements 1.sub.1b, 1.sub.2b, 1.sub.3b . . . 1.sub.10b would be lengthened an equal amount. The amount by which these measurements are changed determines the radius of the resultant curve.

In the selectively steerable distal portion 2704 of the articulating instrument body 2702, the actuators that control the a, b, c and d axis measurements of each section are selectively controlled by the user through the use of a known steering control. Thus, by appropriate control of the a, b, c and d axis measurements, the selectively steerable distal portion 2704 of the articulating instrument body 2702 can be selectively steered or bent. In some embodiments, the steerable portion may be bent a full 180 degrees in any direction.

In the automatically controlled proximal portion 2706, however, the a, b, c and d axis measurements of each section are automatically controlled by an electronic motion controller suited to controlling and actuating based on the type of actuator in use. The motion controller implements the follow the leader algorithm, such as a curve propagation method, to automatically control the shape of the articulating instrument body 2702. To explain how the curve propagation method operates, FIG. 28 shows the wire frame model of a part of the automatically controlled proximal portion 2706 of the articulating instrument body 2702 shown in FIG. 27 passing through a curve C. For simplicity, an example of a two-dimensional curve is shown and only the a and b axes will be considered. In a three-dimensional curve all axes (in the illustrative example, four namely the a, b, c and d axes) would be brought into play.

In FIG. 28, the articulating instrument body 2702 has been maneuvered through the curve C with the benefit of the selectively steerable distal portion 2704 (this part of the procedure is explained in more detail below) and now the automatically controlled proximal portion 2706 resides in the curve. Sections 1 and 2 are in a relatively straight part of the curve C, therefore 1.sub.1a=1.sub.1b and 1.sub.2a=1.sub.2b. However, because sections 3-7 are in the S-shaped curved section, 1.sub.3a<1.sub.3b, 1.sub.4a<1.sub.4b and 1.sub.5a<1.sub.5b, but 1.sub.6a>1.sub.6b, 1.sub.7a>1.sub.7b and 1.sub.8a>1.sub.8b. When the articulating instrument body 2702 is advanced distally by one unit, section 1 moves into the position marked 1′, section 2 moves into the position previously occupied by section 1, section 3 moves into the position previously occupied by section 2, etc. An axial motion transducer may be used to produces a signal indicative of the axial position of the articulating instrument body 2702 with respect to a fixed point of reference and sends the signal to the electronic motion controller. Under control of the electronic motion controller, each time the articulating instrument body 2702 advances one unit, each section in the automatically controlled proximal portion 2706 is signaled to assume the shape of the section that previously occupied the space that it is now in. Therefore, when the articulating instrument body 2702 is advanced to the position marked 1′, 1.sub.1a=1.sub.1b, 1.sub.2a=1.sub.2b, 1.sub.3a=1.sub.3b, 1.sub.4a<1.sub.4b, 1.sub.5a<1.sub.5b, 1.sub.6a<1.sub.6b, 1.sub.7a>1.sub.7b, 1.sub.8a>1.sub.8b, and 1.sub.9a>1.sub.9b, and, when the articulating instrument body 102 is advanced to the position marked 1″, 1.sub.1a=1.sub.1b, 1.sub.2a=1.sub.2b, 1.sub.3a=1.sub.3b, 1.sub.4a=1.sub.4b, 1.sub.5a<1.sub.5b, 1.sub.6a<1.sub.6b, 1.sub.7a <1.sub.7b, 1.sub.8a>1.sub.8b, 1.sub.9a>1.sub.9b, and 1.sub.10a>1.sub.10b. Thus, the S-shaped curve C propagates proximally along the length of the automatically controlled proximal portion 2706 of the articulating instrument body 102. The S-shaped curve appears to be fixed in space, as the articulating instrument body 102 advances distally.

Similarly, when the articulating instrument body 2702 is withdrawn proximally, each time the articulating instrument body 2702 is moved proximally by one unit, each section in the automatically controlled proximal portion 2706 is signaled to assume the shape of the section that previously occupied the space that it is now in. The S-shaped curve propagates distally along the length of the automatically controlled proximal portion 2706 of the articulating instrument body 2702, and the S-shaped curve appears to be fixed in space, as the articulating instrument body 102 withdraws proximally.

Whenever the articulating instrument body 2702 is advanced or withdrawn, the axial motion transducer detects the change in position and the electronic motion controller propagates the selected curves proximally or distally along the automatically controlled proximal portion 2706 of the articulating instrument body 2702 to maintain the curves in a spatially fixed position. This allows the articulating instrument body 102 to move through a tortuous curve without putting unnecessary force on the wall(s) of the pathway being traversed, such as for example, within an organ, about an organ or through the vasculature, or inside the colon.

As used herein a curve, advancing or withdrawing along a curve or path refers not only to a simple curves and paths but also includes complex curves, a series of simple or complex curves, including 3-D space or zones in both medical and industrial environments. Movement, advancement or otherwise propagating along or withdrawing from are also included.

Controlled bending of segments in an articulating instrument using activated polymer electrodes may be performed using a number of techniques. Some of the techniques described herein includes use of a bias element or pre-strain in an instrument, cooperative pairings of activated polymer actuators, voltage control to adjust the amount of deflection induced by an active area and compound actuations realized through the use of multiple active areas, degrees of freedom and compound laminated polymer actuators. Another alternative involves sequential control of multiple active areas to produce a desired curve.

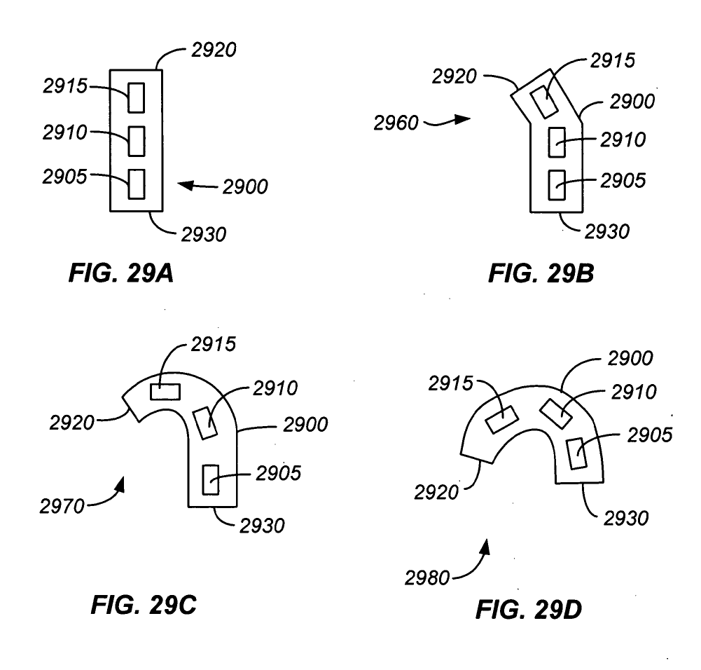

FIGS. 29(a)-(d) illustrate how sequential activation and control of a number of active areas may be used to bend segment 2900. The segment 2900 forms a portion of an articulated instrument or may be a complete instrument. In this illustrative embodiment, the segment 2900 has a distal end 2920, a proximal end 2930 and three active areas 2905, 2910 and 2905. The degree of bending of the segment is controlled by the number of active areas that are actuated. When only active area 2915 is activated, a slight bend 2960 is introduced into the segment (FIG. 29 (a)). Note that when both active areas 2915 and 2910 are activated, segment 2900 forms a bend 2970 that is sharper than bend 2960 sharper (FIG. 29 (c)). When all three active areas 2915, 2910, 2905 are activated, segment 2900 forms an even, sharper bend 2980. While this illustrative embodiment uses three active areas that are aligned generally longitudinally along segment 2900, it is to be appreciated that more, fewer, differently oriented, differently sized, and differently activated active areas may be utilized.

Additionally, the active areas 2915, 2910 and 2905 are illustrated and described as single electrode or as being only single active areas. In some embodiments, the active area may include numbers electrodes and may be able to further subdivide the degree of bending. Consider for example the illustrative case where active area 2910 includes 20 sub-active areas within the larger illustrated area. Each of the sub-active areas are aligned relative to the segment 2900 to bend the segment from the bend 2960 condition to the 2970 bend condition. However, unlike the above described single step of activate active area 2910 to produce bend 2970, the sub-active areas my be activated one at a time to produce intermediate bend conditions between bend 2960 and bend 2970. In another alternative, a controller using an algorithm determines the number/amount etc. of active areas to be activated for a desired curve. In additional embodiments, the use of multiple sub-active areas my be advantageously employed to make the response time more rapid. While desiring not to be bound my theory, there may be polymer actuator configurations that utilize a plurality of sub-active areas to produce a segment with a more rapid response time than a similar segment that only uses a single active area.

While the concept of sequential activation and control is described using a single two-dimensional bend, it is to be appreciated that this concept may be advantageously employed throughout the alternative actuator embodiments described herein for even the most complex shapes. For example, the orientation, size and placement of active areas within embodiments of the compound laminate polymer actuators may also be determined utilizing sequential activation and control. The name of this concept does not imply that actuators may not be activated simultaneously and only sequentially. Sequential refers to the adding more and more actuators until the desired bend, shape or manipulation is achieved. Even adding on more actuators could be done by the controller used to activate the active areas since the bending—active area activation curves will likely be known or sufficiently characterized to allow rapid activation for a desired curve.

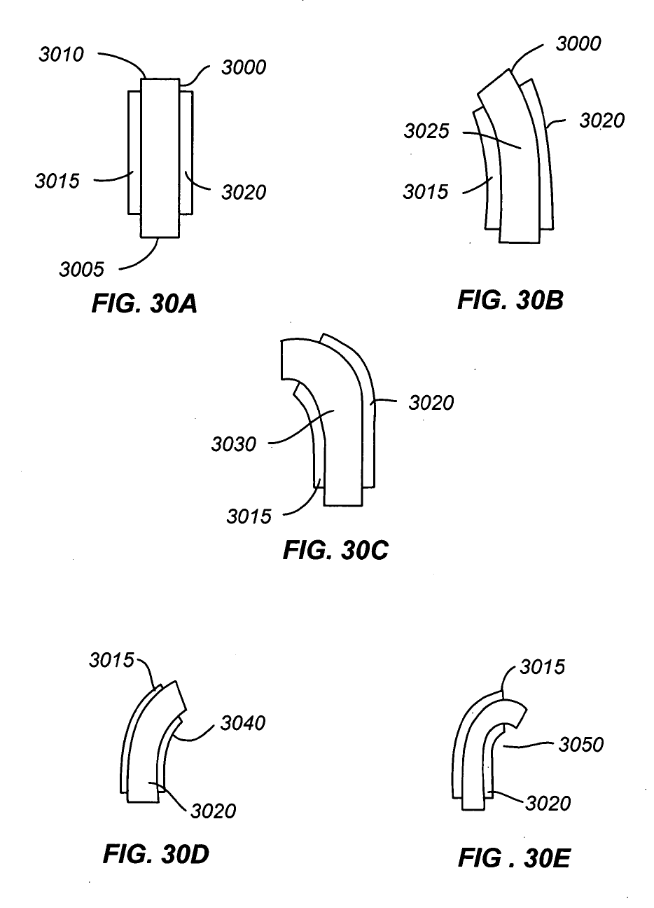

FIG. 30 illustrates a segment 3000 having a distal end 3010 and a proximal end 3005 and active areas or electrodes 3015, 3020. Segment 3000 is specifically designed to bend when one or both of the active areas 3015, 3020 are inactive. For example, FIG. 30(a) illustrates the case where the electrode or electrodes in both active areas 3015, 3020 are activated. The active areas are specifically aligned to utilize polymeric induced deflection to lengthen the polymer along the sides of segment 3000. As a result, the deflection/deformation induced by active area 3015 is balanced or off set by the deflection/deformation induced by active area 3020. Hence, the segment 3000 maintains the straight or linear position shown. Next, consider the case when active area 3015 is inactive. When active area 3015 is not deforming its associated polymer, the polymer on that side (like the polymer associated with active area 3020 on the other side) contracts thereby producing the bend 3025 in segment 3000. In still another embodiment, the active area 3015 may be so configured that reversing the potential applied to active area 3015 actually increases the segment bend to bend 3030. A similar phenomenon is exhibited by active area 3020 to produce bend 3040 (active area 3020 not active) and bend 3050 when the potential on the active area 3020 is reversed. The arrangements and configurations of the active areas to produce the bends 3025, 3040 (inactive state induced bend) may be used independently from the bends 3030 and 3050 produced using reversed potential. In some embodiments, the inactive state induced bend may be used in concert with the reversed potential induced bends.

Embodiments of the electromechanical actuator controlled articulating instruments of the invention may also be advantageously modified to suit uses in a variety of different diagnostic and interventional procedures, including colonoscopy, bronchoscopy, thoracoscopy, laparoscopy and video endoscopy using the principals and concepts described above. Articulating instruments according to embodiments of the present invention may also be used for industrial applications such as inspection and exploratory applications within tortuous regions, e.g., machinery, pipes, difficult to access enclosures and the like.

This invention has been described and specific examples of the invention have been portrayed. The use of those specifics is not intended to limit the invention in any way. For instance, the devices and methods described herein may also be used for non-medically related procedures. It is also contemplated that combinations of features between various examples disclosed above may be utilized with one another in other variations. Additionally, to the extent there are variations of the invention which are within the spirit of the disclosure and yet are equivalent to the inventions found in the claims, it is our intent that this patent will cover those variations as well.

1-20. (canceled)

21. A device, comprising;
a plurality of structural elements coupled together to form an elongate and articulatable instrument; and
a plurality of electroactive polymer actuators coupled to each other and disposed between at least one pair of adjacently disposed structural elements of the plurality of structural elements, wherein the electroactive polymer actuators are configured to be placed in an actuated state to articulate the pair of structural elements relative to one another;
wherein, in the actuated state:
at least one of the electroactive polymer actuators transmits force to another of the electroactive polymer actuators, and
the articulation of the pair of structural elements is based on a cumulative deflection of each of the actuated electroactive polymers.

22. The device of claim 21, wherein the plurality of electroactive polymer actuators are coupled together in a nested configuration.

23. The device of claim 21, wherein the plurality of electroactive polymer actuators have a shape of hollow rolls.

24. The device of claim 23, wherein the plurality of electroactive polymer actuators are coupled together in a concentric configuration.

25. The device of claim 21, wherein the plurality of electroactive polymer actuators are configured to expand or contract along an axial direction of the instrument in the actuated state.

26. The device of claim 21, further comprising a connector disposed between an end portion of a first electroactive polymer actuator of the plurality of electroactive polymer actuators and an end portion of a second electroactive polymer actuator of the plurality of electroactive polymer actuators;
wherein, in an non-actuated state, the end portion of the first electroactive polymer actuator is located at a distal region of the electroactive polymer actuators and the end portion of the second electroactive polymer actuator is located at a proximal region of the electroactive polymer actuators.

27. The device of claim 21, further comprising a connector disposed between an end portion of a first electroactive polymer actuator of the plurality of electroactive polymer actuators and an end portion of a second electroactive polymer actuator of the plurality of electroactive polymer actuators;
wherein, in a non-actuated state, the end portions of the first and second electroactive polymer actuators are both located at either a distal region or a proximal region of the electroactive polymer actuators.

28. The device of claim 21, wherein the plurality of electroactive polymer actuators are configured to be placed in the actuated state in unison.

29. The device of claim 21, wherein the plurality of electroactive polymer actuators are configured to be placed in the actuated state independently of one another.

30. The device of claim 21, wherein the cumulative deflection of the plurality of electroactive polymer actuators is a sum of individual deflections of the plurality of electroactive polymer actuators.

31. The device of claim 21, wherein the instrument comprises a plurality of separate sets of the plurality of electroactive polymer actuators, wherein the sets of actuators are coupled to and disposed between the pair of structural elements.

32. The device of claim 21, wherein the instrument comprises a selectively steerable distal end comprising a plurality of segments, wherein each of the plurality of segments comprises the pair of structural elements and the plurality of electroactive polymer actuators coupled to and disposed between the pair of structural elements.

33. The device of claim 21, wherein the instrument comprises an automatically controllable proximal end comprising a plurality of segments, wherein each of the plurality of segments comprises the pair of structural elements and the plurality of electroactive polymer actuators coupled to and disposed between the pair of structural elements.

34. The device of claim 21, wherein each of the plurality of electroactive polymer actuators comprises at least one pair of electrodes that define an active area for each of the plurality of electroactive polymer actuators, the active area being deformable in response to application of a voltage to the electrodes.

35. The device of claim 34, wherein each of the plurality of electroactive polymer actuators is configured to exhibit an induced strain proportional to a square of an electric field produced by the pair of electrodes in response to application of the electric field to the electroactive polymer actuators.

36. The device of claim 21, wherein deflection of the plurality of electroactive polymer actuators in the actuated state occurs along an axial direction of the instrument.

37. The device of claim 21, wherein the plurality of electroactive polymer actuators are coupled in series between the pair of structural elements.

38. The device of claim 21, wherein the plurality of electroactive polymer actuators are configured to move relative to one another along an axial direction of the instrument.

39. The device of claim 21, wherein the plurality of electroactive polymer actuators are configured to move relative to one another in a telescoping motion.

40. A surgical system, comprising:
an elongate and articulatable surgical instrument, comprising:
a plurality of structural elements coupled together; and
a plurality of electroactive polymer actuators coupled to each other and disposed between at least one pair of adjacently disposed structural elements of the plurality of structural elements, wherein the electroactive polymer actuators are configured to be placed in an actuated state to articulate the pair of structural elements relative to one another;
wherein the plurality of electroactive polymer actuators are coupled together in a configuration such that, in an actuated state, at least one of the electroactive polymer actuators transmits force to another of the electroactive polymer actuators and the articulation of the pair of structural elements is based on a cumulative deflection of each of the actuated electroactive polymers; and
an electronic motion controller operably coupled to the surgical instrument and configured to control actuation of the plurality of electroactive polymer actuators in a state of the surgical instrument being operably coupled to the electronic motion controller.

41. The surgical system of claim 40, wherein the electronic motion controller is configured to actuate the plurality of electroactive polymer actuators in unison.

42. The surgical system of claim 40, wherein the electronic motion controller is configured to actuate the plurality of electroactive polymer actuators independently of one another.
